(19) United States
(12) Patent Application Publication
(10) Pub. No.: US 2026/0002131 A1
(43) Pub. Date: Jan. 1, 2026

(54) CULTURE MEDIUM SUITABLE FOR THE DIFFERENTIATION AND CULTURE OF INTESTINAL ORGANOIDS

(71) Applicant: FRIEDRICH MIESCHER INSTITUTE FOR BIOMEDICAL RESEARCH, Basel (CH)

(72) Inventors: Maurice KAHNWALD, Basel (CH); Prisca LIBERALI, Zürich (CH); Koen OOST, Oberwil (CH)

(73) Assignee: FRIEDRICH MIESCHER INSTITUTE FOR BIOMEDICAL RESEARCH, Basel (CH)

(21) Appl. No.: 18/880,896

(22) PCT Filed: Jul. 6, 2023
(86) PCT No.: PCT/IB2023/056979

§ 371 Date: Jan. 3, 2025

(30) Foreign Application Priority Data
Jul. 8, 2022 (EP) 22183854.3

(51) Int. Cl. C12N 5/071

(52) U.S. Cl. CPC C12N 5/0679; C12N 2500/38; C12N 2501/105; C12N 2501/115; C12N 2501/13; C12N 2501/345; C12N 2501/405; C12N 2501/415; C12N 2501/727; C12N 2513/00; C12N 2533/90

(57) ABSTRACT

The present application relates to a culture medium for the culture of intestinal organoids characterized in that it comprises neuregulin 1 (NRG1) and all-trans retinoic acid (atRA). It also relates to a method using this medium to culture intestinal organoids.

### FIELD OF THE INVENTION

The present invention relates to a culture medium suitable for the long- or short-term in vitro culture of intestinal organoids cells.

### BACKGROUND OF THE INVENTION

The intestinal tract is an organ having the largest area of contact with the external environment in the human body and has a function indispensable for maintaining life, such as digestion and absorption. Most of intestinal function is carried by the intestinal epithelium covering an inner layer thereof. The intestinal epithelium is composed of two compartments: a villus consisting of three differentiated cells (mucus producing cells, absorptive epithelial cells, and endocrine cells) and a crypt mainly consisting of undifferentiated proliferating cells. In the small intestinal crypt, Paneth cells that produce antimicrobial peptides are present at the bottom of the crypt. Recently, molecular genetic cell lineage analysis has revealed that Lgr5-positive cells (also called “crypt base columnar (CBC) cells”) sandwiched between Paneth cells are intestinal epithelial stem cells. Lgr5-positive intestinal epithelial stem cells produce progenitor cells called transit amplifying cells, but these progenitor cells have no permanent self-renewal ability and are also limited to 1 to 3 lines of differentiation potential. The transit amplifying cells differentiate with 2 to 4 divisions in the crypts and terminally differentiate in the villi. These differentiated cells detach at the apex of villi and die by apoptosis. The intestinal epithelium is a rapidly metabolizing tissue and migrates from the crypt stem cells to the apex of the villi in 4 to 5 days. Unlike other differentiated cells, Paneth cells migrate to the bottom of the crypt under differentiation and have a long cell life of 2 months.

The self-renewal mechanism of intestinal epithelial stem cells is known to be controlled by Wnt signal and bone morphogenetic protein (BMP) signal from the results of several genetically modified mice. Intestinal epithelial-specific knockout mice of adenomatous polyposis coli (APC) which is a suppressor molecule of Wnt signal show intestinal epithelial cell hyperproliferation and adenomas formation. In addition, ectopic crypt formation was observed in mice overexpressing Noggin, which is an inhibitory protein of BMP, in intestinal epithelial cells, and therefore it has been suggested that the BMP signal inhibitively acts on intestinal epithelial stem cells.

In addition, long-term culture of intestinal epithelial cells has been impossible for a long time. This is considered to be due to the fact that the growth factor necessary for the maintenance of intestinal epithelial stem cells was unknown. In recent years, successful long-term maintenance of intestinal epithelial stem cells has been achieved by adhering intestinal epithelial stem cells onto an extracellular matrix and culturing the cells in the presence of a cell culture medium containing a basic medium for animal or human cells to which a BMP inhibitor, a mitogenic growth factor, and a Wnt agonist are added. Another tool for the study of the intestinal tract is the study of intestinal organoids. Organoids are tiny, self-organized three-dimensional tissue cultures that are derived from stem cells. Such cultures can be crafted to replicate much of the complexity of an organ, or to express selected aspects of it like producing only certain types of cells. Organoid formation generally requires culturing the stem cells or progenitor cells in an organ/tissue specific medium.

### SUMMARY OF THE INVENTION

Through their work, the present inventors surprisingly succeeded in defining a culture medium suitable for the long- or short-term in vitro culture of intestinal organoids cells.

The claims thus encompass a culture medium for the culture of intestinal organoids characterized in that it comprises neuregulin 1 (NRG1) and all-trans retinoic acid (atRA). This culture medium of the invention is suitable for the culture of small intestinal organoids, colon organoids, colorectal cancer organoids, Crohn's disease organoids or Ulcerative Colitis organoids.

The concentration of NRG1 in the medium can be between 1 ng/ml and 100 ng/ml. The concentration of atRA in the medium can be between 10 nM and 10 μM. In some embodiments, the culture medium of the invention is free of animal serum. In some embodiments, the culture medium of the invention further comprises any of R-Spondin, recombinant Noggin, NRG1, IGF1, FGF2 and Gastrin. All of these factors, NRG1 and atRA included, can be human or mouse, as well as recombinant or purified natural proteins. The culture medium of the invention can also comprise Wnt NGS (Next Generation Surrogate). The claims also encompass a composition comprising the medium of the invention together with an extracellular matrix or a 3D matrix that mimics the extracellular matrix by its interactions with cellular membrane proteins. Such a 3D matrix extracellular matrix can be synthetic hydrogels or Matrigel™. Alternatively, among others, UltriMatrix (Culturex Ultimatrix RGF basement membrane extract) and BME (Cultrex Reduced Growth Factor Basement Membrane Extract, Type 2, Pathclear), as well as Collagen-1 or Fibronectin can be used. In some embodiments, the extracellular matrix or 3D matrix is washed away after an initial period.

The claims further encompass the use of a medium of the invention, or a composition of the invention, for the culture of intestinal organoids.

The claims further encompass a method of generating intestinal organoids comprising the steps of providing a suspension of single cells or fragments of intestinal tissues incubating said suspension of single cells or fragments of intestinal tissue with a first medium for at least one day, and thereafter exchanging said first medium with a medium of the invention as described above. In some embodiments, the first medium can comprise atRA at a concentration of 10 nM. The single cells or fragments of intestinal tissues used in the method of the invention can be cells, or tissue fragments, obtained from small intestine, colon, colorectal cancer, Crohn's disease tissue, Ulcerative Colitis tissue, or can be definitive endoderm cells.

In the method of the invention, the first medium can comprise an inhibitor of the Rho-associated kinase (ROCK), for instance the ROCK inhibitor Y27632. In the method of the invention, the first medium can comprise an inhibitor of the Activin/NODAL/TGF-β pathway, for instance the inhibitor A 83-01.

In some embodiments of the method of the invention, the cells can be incubated in the first medium for at least three days.

The first and second media of the method of the invention can be adapted to the nature of the single cells or fragments of intestinal tissues used in said method of the invention. For instance, for small intestine cells and/or tissue fragments, an appropriate concentration for NRG1 in the first-medium is 1 ng/ml and no atRA, whereas the concentration for NRG1 and atRA in the second medium, i.e. medium, of the invention, is 10 ng/ml and 2.5 μM, respectively. For cells and tissue fragments from the colon, an appropriate concentration for NRG1 in the first medium is 1 ng/ml and no atRA and a concentration of 10 ng/ml and 10 nM for NRG1 and atRA, respectively, in the second-medium.

The claims also encompass intestinal organoids obtained using the method of the invention.

### BRIEF DESCRIPTION OF THE DRAWINGS

The following drawings are illustrative of embodiments of the invention and are not meant to limit the scope of the invention as encompassed by the claims.

FIG. 1. Image based medium screen for human large and small intestinal organoids from single cells towards homeostatic mature organoids. A) Schematic representation of the workflow, where 100 single cells per well are initially seeded from trypsinized mature colonic organoids. For the initial medium 2 different EGF/NRG1 combinations were tested. Medium was changed at day 4 to one of 16 different media varying in their EGF, hNRG1, WNT-NGS concentration, totaling 32 combinations. Organoids were fixed at day 10 and 5848 organoids were segmented based on maximum intensity projections of the DAPI-channel (RDCNET ref), from which shape features were extracted. B) Force directed layout (FDL, Eccentricity: left and log₁₀ (Area): right) of 5848 human colon organoids computed based on morphological organoid features. C) PhenoGraph-clusters represented in the FDL highlighting subpopulations (cluster 0 till 15). Representative organoid of three clusters exemplified below (cluster 0: blue, cluster 7: salmon, and cluster 4: dark-green). D) Heatmap quantifying performance of all media 32 combinations tested in human colon organoids. Shown are scores for important parameters in assessing the performance of the medium. Scores are normalized based on the relative performance of the media (0: worst-performing, 1: best performing). See Table 1 for detailed medium compositions. Condition 2.3.3 (green square) is the selected condition for future experiments. E) Force directed layout (FDL, Eccentricity: left and log₁₀ (Area): right) of 5533 human small intestinal organoids computed based on morphological organoid features. F) PhenoGraph-clusters represented in the FDL highlighting subpopulations (cluster 0 till 15). Representative organoid of three clusters exemplified below (cluster 3; light-orange, cluster 11; light-brown, and cluster 4; dark-green). G) Heatmap quantifying performance of 8 combinations tested in human small intestinal organoids, similar to D. Compared to human colonic organoids, small intestinal organoids were not dependent on WNT-NGS after day 4. See Table 1 for detailed medium compositions. Condition 2.3.1 (green square) is the selected condition for future experiments. H) Brightfield images of human healthy colonic and colorectal cancer (CRC) organoid lines from the same patient in 384-well plates at the fixation timepoints day 2, 4, 6, 8, 10, and 13. For both organoid time courses the selected medium composition has been used (see FIG. 1D). 1) Antibody staining for major mature cell types in mature colon (upper pane) and small intestine (lower panel) organoids, grown in selected medium composition (see FIG. 1D, 1G, respectively). Panels from left to right: stem cells (OLFM4; green) and enterocytes (FABP1; red), goblet cells (MUC2; red), transient amplifying cells (NUSAP1; red), tuft cells (POU2F3; red), M-cells (RANKL; red), proliferation marker (KI67; red), enteroendocrine cells (TPH1; red), deep secretory cells (REG4; red), and Paneth cells (LYZ1; red). Nuclear marker in all organoids is DAPI (white).

Scale bars: 100 μm in I, 50 μm in C, F.

FIG. 2 Comparison between different media regimes from single cells towards mature organoids. A) Brightfield images demonstrating human colon organoid outgrowth efficiency for the same organoid line in different media regimes at day 10. 250 single cells have been seeded at day 0. B) Quantified human colon organoid outgrowth efficiency for different media at day 4 and 8. C-D) Human colonic organoid imaging time course (i.e., day 2, 4, 6, 8, and 10) in 4 different media regimes all stained for (C) stem cell marker OLFM4 (green) and enterocyte marker FABP1 (red) or (D) goblet cell marker MUC2 (red). E) Fluorescent images capturing organoid development from different biopsy locations (indicated on the left in each panel) of the colon using the developed medium regime at day 2, 4, 6, 8, 10 and 13. Each developmental trajectory represents a different patient, showing enteroendocrine (TPH1; magenta), goblet (MUC2; red), and transient amplifying cells (NUSAP1; green). Nuclear marker in all organoids is DAPI (white). Scale bars: 50 μm.

FIG. 3 Pseudotime of intestinal organoids reveals the transition from regeneration towards a homeostatic equilibrium by using the FMI-regime. A) Schematic overview of human colon organoid seeding and timepoint sampling. B) After individual organoid segmentation (FIG. 1A) features are used to order individual organoids based on their shape-features in pseudotime (palantir, Setty et al., 2019), approximating maturation progression. C) Pseudotime of organoid development binned into 10 equal bins for DAPI, OLFM4, FABP1, MUC2, YAP1, and TPH1. The fluorescent images (inferno LUT) are representative for each bin. On the right side, mean intensities of the respective markers and important shape features over all imaged organoids are plotted against the 10 pseudotime bins.

FIG. 4 single cell RNA sequencing timecourse of human colon and small intestinal organoid regeneration. A-D) t-distributed stochastic neighbor embedding (t-SNE) projection color coded for experimental time, and B-E) cell types. Left colon, right small intestine. C-F) Relative expression of known cell type marker genes in regenerative and mature cell types highlighted in t-SNE maps.

FIG. 5 Individual show cases for organoid cultures using the FMI-regime. A) One week old human organoids generated from fresh biopsies of inflammatory bowel disease (IBD) patients. Brightfield images of Crohn's (left), and healthy (middle) plus diseased (right) colon from an Ulcerative Colitis patient. B-C) Brightfield images of mouse small intestine organoids plated in (B) different densities at day 7. C) Brightfield zoom-ins onto the same demonstrating organoid development from single cell towards budding organoids using the FMI-regime. D) Schematic overview of the outgrowth of either Lgr5-negative or positive mouse colon cells using the FMI-regime. Adapted from Serra et al., 2019. E) Representative fluorescent images for Lgr5-positive (green) (upper panel) and negative (lower panel) organoid development. F) Demonstrating the proliferative regions (Ki67=red) and stem cell (Lgr5=green) overtime. G) Three-panels showing besides Lgr5 (green) the Wnt-target gene Sox9 (upper; red), MUC2 (middle; goblet cell marker), and FABP2 (lower; enterocyte marker). Nuclear marker in all organoids is DAPI (blue).

### DETAILED DESCRIPTION OF THE INVENTION

Through their work, the present inventors surprisingly succeeded in defining a culture medium suitable for the long- or short-term in vitro culture of intestinal organoids cells.

The claims thus encompass a culture medium for the culture of intestinal organoids characterized in that it comprises neuregulin 1 (NRG1) and all-trans retinoic acid (atRA). This culture medium of the invention is suitable for the culture of small intestinal organoids, colon organoids, colorectal cancer organoids, Crohn's disease organoids or Ulcerative Colitis organoids.

The concentration of NRG1 in the medium can be between 1 ng/ml and 100 ng/ml. The concentration of atRA in the medium can be between 10 nM and 10 μM. In some embodiments, the culture medium of the invention is free of animal serum. In some embodiments, the culture medium of the invention further comprises any of R-Spondin, recombinant Noggin, NRG1, IGF1, FGF2 and Gastrin. All of these factors, NRG1 and atRA included, can be human or mouse, as well as recombinant or purified natural proteins. The culture medium of the invention can also comprise Wnt NGS (Next Generation Surrogate). The claims also encompass a composition comprising the medium of the invention together with an extracellular matrix or a 3D matrix that mimics the extracellular matrix by its interactions with cellular membrane proteins. Such a 3D matrix extracellular matrix can be synthetic hydrogels or Matrigel™. Alternatively, among others, UltriMatrix (Culturex Ultimatrix RGF basement membrane extract) and BME (Cultrex Reduced Growth Factor Basement Membrane Extract, Type 2, Pathclear), as well as Collagen-1 or Fibronectin can be used. In some embodiments, the extracellular matrix or 3D matrix is washed away after an initial period.

The claims further encompass the use of a medium of the invention, or a composition of the invention, for the culture of intestinal organoids.

The claims further encompass a method of generating intestinal organoids comprising the steps of providing a suspension of single cells or fragments of intestinal tissues incubating said suspension of single cells or fragments of intestinal tissue with a first medium for at least one day, and thereafter exchanging said first medium with a medium of the invention as described above. In some embodiments, the first medium can comprise atRA at a concentration of 10 nM. The single cells or fragments of intestinal tissues used in the method of the invention can be cells, or tissue fragments, obtained from small intestine, colon, colorectal cancer, Crohn's disease tissue, Ulcerative Colitis tissue, or can be definitive endoderm cells.

In the method of the invention, the first medium can comprise an inhibitor of the Rho-associated kinase (ROCK), for instance the ROCK inhibitor Y27632. In the method of the invention, the first medium can comprise an inhibitor of the Activin/NODAL/TGF-β pathway, for instance the inhibitor A 83-01.

In some embodiments of the method of the invention, the cells can be incubated in the first medium for at least three days.

The first and second media of the method of the invention can be adapted to the nature of the single cells or fragments of intestinal tissues used in said method of the invention. For instance, for small intestine cells and/or tissue fragments, an appropriate concentration for NRG1 in the first-medium is 1 ng/ml and no atRA, whereas the concentration for NRG1 and atRA in the second medium, i.e. medium, of the invention, is 10 ng/ml and 2.5 μM, respectively. For cells and tissue fragments from the colon, an appropriate concentration for NRG1 in the first medium is 1 ng/ml and no atRA and a concentration of 10 ng/ml and 10 nM for NRG1 and atRA, respectively, in the second-medium.

The claims also encompass intestinal organoids obtained using the method of the invention.

The following definitions are provided to facilitate understanding of certain terms used throughout this specification.

As used herein, the term “totipotent stem cells” (also known as omnipotent stem cells) are stem cells that can differentiate into embryonic and extraembryonic cell types. Such cells can construct a complete, viable, organism. These cells are produced from the fusion of an egg and sperm cell. Cells produced by the first few divisions of the fertilized egg are also totipotent.

As used herein, the term “pluripotent stem cells (PSCs),” also commonly known as PS cells, encompasses any cells that can differentiate into nearly all cells, i.e., cells derived from any of the three germ layers (germinal epithelium), including endoderm (interior stomach lining, gastrointestinal tract, the lungs), mesoderm (muscle, bone, blood, urogenital), and ectoderm (epidermal tissues and nervous system). PSCs can be the descendants of totipotent cells, derived from embryonic stem cells (including embryonic germ cells) or obtained through induction of a non-pluripotent cell, such as an adult somatic cell, by forcing the expression of certain genes.

As used herein, the term “induced pluripotent stem cells (iPSCs),” also commonly abbreviated as iPS cells, refers to a type of pluripotent stem cells artificially derived from a normally non-pluripotent cell, such as an adult somatic cell, by inducing a “forced” expression of certain genes.

As used herein, the term “embryonic stem cells (ESCs),” also commonly abbreviated as ES cells, refers to cells that are pluripotent and derived from the inner cell mass of the blastocyst, an early-stage embryo. For purpose of the present invention, the term “ESCs” is used broadly sometimes to encompass the embryonic germ cells as well.

As used herein, the term “precursor cell” encompasses any cells that can be used in methods described herein, through which one or more precursor cells acquire the ability to renew itself or differentiate into one or more specialized cell types. In some aspects, a precursor cell is pluripotent or has the capacity to becoming pluripotent. In some aspects, the precursor cells are subjected to the treatment of external factors (e.g., growth factors) to acquire pluripotency. In some aspects, a precursor cell can be a totipotent (or omnipotent) stem cell; a pluripotent stem cell (induced or non-induced); a multipotent stem cell; an oligopotent stem cells and a unipotent stem cell. In some aspects, a precursor cell can be from an embryo, an infant, a child, or an adult. In some aspects, a precursor cell can be a somatic cell subject to treatment such that pluripotency is conferred via genetic manipulation or protein/peptide treatment.

In developmental biology, cellular differentiation is the process by which a less specialized cell becomes a more specialized cell type. As used herein, the term “directed differentiation” describes a process through which a less specialized cell becomes a particular specialized target cell type. The particularity of the specialized target cell type can be determined by any applicable methods that can be used to define or alter the destiny of the initial cell. Exemplary methods include but are not limited to genetic manipulation, chemical treatment, protein treatment, and nucleic acid treatment.

As used herein, the term “cellular constituents” are individual genes, proteins, mRNA expressing genes, and/or any other variable cellular component or protein activities such as the degree of protein modification (e.g., phosphorylation), for example, that is typically measured in biological experiments (e.g., by microarray or immunohistochemistry) by those skilled in the art. Significant discoveries relating to the complex networks of biochemical processes underlying living systems, common human diseases, and gene discovery and structure determination can now be attributed to the application of cellular constituent abundance data as part of the research process. Cellular constituent abundance data can help to identify biomarkers, discriminate disease subtypes and identify mechanisms of toxicity.

As described herein, methods and systems are established using a temporal series of growth factor manipulations to mimic embryonic intestinal development in culture. In particular, methods and systems are established to direct in vitro differentiation of PSCs, both human embryonic stem cells (hESC) and induced pluripotent stem cells (iPSC), into intestinal tissue

The generation of gastric and small intestinal organoids from pluripotent stem cells (PSCs) has revolutionized the study human gastrointestinal (GI) development and disease. However, efforts to generate large intestinal organoids have lagged behind, in part due to a robust molecular understanding of posterior gut tube development.

In certain embodiments of the invention, “about” or “approximately” refers to a number that varies by up to 5%, or in other embodiments up to 10%, and in other embodiments up to 25%, from the number being referred to.

As used herein, the term “serum-free” refers to the fact that the medium contains essentially no serum. In certain embodiments, there is 0% (completely free), or less than about 0.001%, 0.005%, 0.01%, 0.025%, 0.05%, 0.1%, 1.0%, or 10.0% total serum in the subject medium. The most common types of serums include: various forms of bovine serum (calf serum, fetal bovine serum, bovine calf serum, donor bovine calf serum, newborn bovine calf serum, etc.), horse serum and human serum.

“Chemically defined” means the structures, chemical formulae, and the percentage of the various individual components within a chemical composition are known or can be defined. Various tissue extracts, such as bovine pituitary extracts, are not chemically defined, at least partly because not all individual components of the extract are known. For those known components, the amount and the relative percentages of the various components could (and usually do) vary from one batch to another.

This is partly caused by the fact that individual animals may have inherently different levels of various chemical compositions, even in the same tissue, depending on such factors a general health, nutrition, mood, pathological infections, trauma, etc.

In certain embodiments, the medium of the instant invention does not contain any animal serum products prepared for tissue culture purposes. Nor does it contain any tissue extracts with unknown/undefined chemical components. Instead, all essential components necessary to support the desired growth/proliferation of desired cell types are chemically defined. Most, if not all, of these individual components can be readily purchased as commercial products from various venders, such as Sigma-Aldrich Corp. (St. Louis, MO), GIBCO-Invitrogen Corp. (Carlsbad, CA); Calbiochem, and/or BD Biosciences (San Jose, CA), etc.

In certain other embodiments, the presence in the subject medium of serum and/or tissue extracts, especially in trace amounts, would not substantially interfere with the characteristics of the medium.

The invention also provides a method to carry out a pharmaceutical/biotechnology product discovery and development, comprising of creation of intestinal organoid models derived from various cell types isolated and propagated using the media and methods of the instant invention, and screening for drug molecule or lead compound libraries in order to identify molecules that affects them.

The phrases “cell culture medium,” “culture medium” (plural “media” in each case) and “medium formulation” refer to a nutritive solution for cultivating cells and may be used interchangeably.

The cell culture media of the present invention are aqueous-based (but can be reconstituted from dry powder and/or frozen components), comprising a number of ingredients in a solution of, preferably deionized and/or distilled, water.

The term “ingredient” refers to any compound, whether of chemical or biological origin, that can be used in cell culture media to maintain or promote the growth of proliferation of cells. The terms “component,” “nutrient” and ingredient” can be used interchangeably and are all meant to refer to such compounds. Typical ingredients that are used in cell culture media include amino acids, salts, metals, sugars, lipids, nucleic acids, hormones, vitamins, fatty acids, proteins and the like. Other ingredients that promote or maintain cultivation of cells ex vivo can be selected by those of skill in the art, in accordance with the particular need.

By “cell culture” or “culture” is meant the maintenance of cells in an artificial, in vitro environment. It is to be understood, however, that the term “cell culture” is a generic term and may be used to encompass the cultivation not only of individual cells, but also of tissues, organs, organ systems or whole organisms, for which the terms “tissue culture”, “organ culture”, “organ system culture”, “organoid culture” or “organotypic culture” may occasionally be used interchangeably with the term “cell culture.”

Certain cells, such as human cells must have adequate amounts of 9 amino acids to survive. These so called “essential” amino acids cannot be synthesized from other precursors. However, cysteine can partially meet the need for methionine (they both contain sulfur), and tyrosine can partially substitute for phenylalanine. Such essential amino acids include: Histidine, Isoleucine, Leucine, Lysine, Methionine (and/or cysteine), Phenylalanine (and/or tyrosine), Threonine, Tryptophan, and Valine. In certain embodiments, only Histidine, Isoleucine, Leucine, Lysine, Threonine, Tryptophan, and Valine are included.

Some or all of the ingredients, when admixed together in solution, can form a “basal medium.” To this basal medium, other components, such as at least one nucleotide synthesis and/or salvage pathway precursors (e.g. hypoxanthine), epidermal growth factor (EGF), agents increasing intracellular cyclic adenosine monophosphate (cAMP) levels, and antioxidants, can be added to formulate the complete culture media of the present invention. These latter added components, such as EGF and the cAMP-increasing agent(s) may be added to freshly formulated basal medium, or they may be admixed as in a stock solution stored frozen, preferably at about −20° C. to about −70° C., until being added to basal medium to formulate the complete medium of the present invention.

To the extent that components do not substantially affect the performance of the medium in terms of culture of intestinal organoids, the subject medium may in certain embodiments include and tolerate the presence of one or more of such components.

One or more components of the medium may also be substituted by other chemicals of similar properties when necessary. Such modified medium without one or more non-essential/unnecessary components are within the scope of the invention. Similarly, a skilled artisan could also determine the optimal level of any given component for a particular cell type, by, for example, testing a range of concentrations (e.g., 10%, 25%, 50%, 75%, 100%, 2-, 5-, 10-, 20-, 50-, 100-, 200-, 500-, 1000-fold higher, or 10%, 25%, 50%, 75%, 100%, 2-, 5-, 10-, 20-, 50-, 100-, 200-, 500-, 1000-fold lower) for each listed component based on or starting from the listed concentration of that particular component. Some components have a listed range of concentrations. The proper or optimal concentration for any particular cell types can also be determined similarly starting from the listed concentration. In doing such tests, initial broad-range concentration tests may be narrowed down later based on the outcomes of the initial experiments. For example, for an initial test, the concentration of one component of interest may be changed to 10⁻³, 10⁻², 10⁻¹, 10-fold, 100-fold, and 1000-fold of the initial concentration. If the 10⁻² test still supports the desired growth, while 10⁻³ fails to, then the 10-fold concentration difference between 10⁻² and 10⁻³ may be further explored in the second round of test to pin-point the best ranges. Thus, media so optimized for specific cell types are also within the scope of the instant invention. As will be readily apparent to one of ordinary skill in the art, the concentration of a given ingredient can be increased or decreased beyond the range disclosed and the effect of the increased or decreased concentration can be determined using only routine experimentation. The optimization of the present media formulations for any specific cell type can be carried out using approaches described by Ham (Ham, Methods for Preparation of Media, Supplements and Substrata for Serum-Free Animal Culture, Alan R. Liss, Inc., New York, pp. 3-21, 1984) and Waymouth (Waymouth, C, Methods for Preparation of Media, Supplements and Substrata for Serum-Free Animal Culture, Alan R. Liss, Inc., New York, pp. 23-68, 1984). The optimal final concentrations for medium ingredients are typically identified either by empirical studies, in single component titration studies, or by interpretation of historical and current scientific literature. In single component titration studies, using animal cells, the concentration of a single medium component is varied while all other constituents and variables are kept constant and the effect of the single component on viability, growth or continued health of the animal cells is measured.

It will be understood that certain factors, vitamins and hormones listed herein can exist in different forms, as known in the art (e.g., different naturally occurring or non-naturally occurring forms), and can be used as substitutes for one another. It will also be appreciated that where the instant application discloses a vitamin or hormone, the invention should be understood to encompass embodiments in which any form of such vitamin or hormone having similar biological activity (or compound(s) that can be modified or metabolized in cell culture medium or intracellularly to provide a biologically active form) is used in the inventive media and/or method(s).

The medium ingredients can be dissolved in a liquid carrier or maintained in dry form. If dissolved in a liquid carrier at the preferred concentrations shown above (i.e., a “1× formulation”), the pH of the medium should be adjusted to about 7.0-7.6, for instance about 7.1-7.5, or about 7.2-7.4. The osmolarity of the medium could also be adjusted to about 275-350 mOsm, for instance about 285-325 mOsm, or about 280-310 mOsm. The type of liquid carrier and the method used to dissolve the ingredients into solution vary and can be determined by one of ordinary skill in the art with no more than routine experimentation. Typically, the medium ingredients can be added in any order.

A cell culture medium is composed of a number of ingredients and these ingredients vary from one culture medium to another. A “1× formulation” is meant to refer to any aqueous solution that contains some or all ingredients found in a cell culture medium at working concentrations. The “1× formulation” can refer to, for example, the cell culture medium or to any subgroup of ingredients for that medium. The concentration of an ingredient in a 1× solution is about the same as the concentration of that ingredient found in a cell culture formulation used for maintaining or cultivating cells in vitro. A cell culture medium used for the in vitro cultivation of cells is a 1× formulation by definition. When a number of ingredients are present, each ingredient in a 1× formulation has a concentration about equal to the concentration of those ingredients in a cell culture medium. For example, RPMI-1640 culture medium contains, among other ingredients, 0.2 g/L L-arginine, 0.05 g/L L-asparagine, and 0.02 g/L L-aspartic aced. A “1× formulation” of these amino acids contains about the same concentrations of these ingredients in solution. Thus, when referring to a “1× formulation,” it is intended that each ingredient in solution has the same or about the same concentration as that found in the cell culture medium being described. The concentrations of ingredients in a 1× formulation of cell culture medium are well known to those of ordinary skill in the art. See Methods for preparation of media, Supplements and Substrate For Serum-Free Animal Cell Culture Allen R. Liss, N. Y. (1984). The osmolality and/or pH, however, may differ in a 1× formulation compared to the culture medium, particularly when fewer ingredients are contained in the 1× formulation.

A “10× formulation” is meant to refer to a solution wherein each ingredient in that solution is about 10 times more concentrated than the same ingredient in the cell culture medium. For example, a 10× formulation of RPMI-1640 culture medium may contain, among other ingredients, 2.0 g/L L-arginine, 0.5 g/L L-asparagine, and 0.2 g/L L-aspartic acid (compare 1× formulation, above). A “10× formulation” may contain a number of additional ingredients at a concentration about 10 times that found in the 1× culture medium. As will be readily apparent, “25× formulation”, “50× formulation”, “100× formulation, “500× formulation”, and “1000× formulation” designate solutions that contain ingredients at about 25-, 50-, 100-, 500-, or 1000-fold concentrations, respectively, as compared to a 1× cell culture medium. Again, the osmolality and pH of the media formulation and concentrated solution may vary. Preferably, the solutions comprising ingredients are more concentrated than the concentration of the same ingredients in a 1× media formulation. The ingredients can be 10-fold more concentrated (10× formulation), 25-fold more concentrated (25× formulation), 50-fold more concentrated (50× formulation), or 100-fold more concentrated (100× formulation). More highly concentrated formulations can be made, provided that the ingredients remain soluble and stable. See e.g. U.S. Pat. No. 5,474, which is directed to methods of solubilizing culture media components at high concentrations.

If the media ingredients are prepared as separate concentrated solutions, an appropriate (sufficient) amount of each concentrate is combined with a diluent to produce a 1× medium formulation. Typically, the diluent used is water but other solutions including aqueous buffers, aqueous saline solution, or other aqueous solutions may be used according to the invention.

The culture media of the present invention are typically sterilized to prevent unwanted contamination. Sterilization may be accomplished, for example, by filtration through a low protein-binding membrane filter of about 0.1-1.0 μm pore size (available commercially, for example, from Millipore, Bedford, Mass.) after admixing the concentrated ingredients to produce a sterile culture medium. Alternatively, concentrated subgroups of ingredients may be filter-sterilized and stored as sterile solutions. These sterile concentrates can then be mixed under aseptic conditions with a sterile diluent to produce a concentrated 1× sterile medium formulation. Autoclaving or other elevated temperature-based methods of sterilization are not favored, since many of the components of the present culture media are heat labile and will be irreversibly degraded by temperatures such as those achieved during most heat sterilization methods.

Many tissue culture media typically contain one or more antibiotics, which are not necessary for cell growth/proliferation per se, but are present to inhibit the growth of other undesirable microbes, such as bacteria and/or fungi. Antibiotics are natural chemical substances of relatively low molecular weight produced by various species of microorganisms, such as bacteria (including Bacillus species), actinomycetes (including Streptomyces) and fungi that inhibit growth of or destroy other microorganisms. Substances of similar structure and mode of action may be synthesized chemically, or natural compounds may be modified to produce semi-synthetic antibiotics. These biosynthetic and semi-synthetic derivatives are also effective as antibiotics. The major classes of antibiotics are: (1) the β-lactams, including the penicillins, cephalosporins and monobactams; (2) the aminoglycosides, e.g., gentamicin, tobramycin, netilmycin, and amikacin; (3) the tetracyclines; (4) the sulfonamides and trimethoprim; (5) the fluoroquinolones, e.g., ciprofloxacin, norfloxacin, and ofloxacin; (6) vancomycin; (7) the macrolides, which include for example, erythromycin, azithromycin, and clarithromycin; and (8) other antibiotics, e.g., the polymyxins, chloramphenicol and the lincosamides. Antibiotics accomplish their anti-bacterial effect through several mechanisms of action which can be generally grouped as follows: (1) agents acting on the bacterial cell wall such as bacitracin, the cephalosporins, cycloserine, fosfomycin, the penicillins, ristocetin, and vancomycin; (2) agents affecting the cell membrane or exerting a detergent effect, such as colistin, novobiocin and polymyxins; (3) agents affecting cellular mechanisms of replication, information transfer, and protein synthesis by their effects on ribosomes, e.g., the aminoglycosides, the tetracyclines, chloramphenicol, clindamycin, cycloheximide, fucidin, lincomycin, puromycin, rifampicin, other streptomycins, and the macrolide antibiotics such as erythromycin and oleandomycin; (4) agents affecting nucleic acid metabolism, e.g., the fluoroquinolones, actinomycin, ethambutol, 5-fluorocytosine, griseofulvin, rifamycins; and (5) drugs affecting intermediary metabolism, such as the sulfonamides, trimethoprim, and the tuberculostatic agents isoniazid and para-aminosalicylic acid. Some agents may have more than one primary mechanism of action, especially at high concentrations. In addition, secondary changes in the structure or metabolism of the bacterial cell often occur after the primary effect of the antimicrobial drug.

Thus for convenience and other practical reasons, the subject media may be additionally supplemented by one or more antibiotics or other substances that inhibit the growth/proliferation of undesirable bacteria/fungi/virus. In other embodiments, however, the subject medium may be free of any antibiotics to ensure optimum growth of primary cells. Extra care should be taken when handling cells growing in antibiotic-free medium in order to avoid possible contamination.

The medium of the instant invention can be made from individual components separately purchased from various chemical vendors. Alternatively, certain commercial medium may be conveniently mixed and supplemented by additional components for make the subject medium. The invention thus provides methods of making a tissue culture medium comprising supplementing a commercially available cell culture medium or mixture of two or more such media by adding one or more components disclosed herein

The invention provides a tissue culture medium for intestinal organoids comprising the components sufficient the growth of cells. The composition of these media may be varied. For example, the concentration of any of the components may be independently varied by up to 10%, 20%, 30%, 40%, or 50%, or by up to a factor of up to 2-3 fold, relative to the original concentrations. In one embodiment, the concentrations of each of the components vary by not more than 10% from the listed value. In one embodiment, the concentrations of each of the components vary by not more than 25% from the listed value. Unless otherwise indicated, as used herein, variation by up to X % means variation by ±X % with respect to the listed value. For example, if the listed value is 100 ng/ml, variation by 25% means that the value can range between 75 ng/ml and 125 ng/ml (i.e., 75-125 ng/ml). Unless otherwise indicated, where a range of values is disclosed, endpoints are included within the range. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate to any intervening value or range defined by any two values in the series, where the lowest value may be taken as a minimum and the greatest value may be taken as a maximum. For any embodiment of the invention in which a value is prefaced by the term “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”.

It will be appreciated that certain of the components may be provided as salts, esters, biologically active metabolites or derivatives, or as precursors that are metabolized, processed, or broken down by the cell or in the medium to yield a biologically active form of certain of the components disclosed herein. “Biologically active” in this context refers to the ability of the component to exert its desired effect on a cell when present in a cell culture medium.

The medium of the instant invention may be liquid or solid powder, or a combination of both. The liquid form may be a complete medium, which contains all the components sufficient to support the growth/proliferation of the target cells. Alternatively, the liquid media may be stored as separate packages, such that each individual package may be stored at its appropriate conditions (temperature, humidity, etc.). For example, most of the components, if desired to be in a medium of the instant invention, can be pre-dissolved in a single solution and stored at appropriate conditions (e.g. 4° C. in a dark and dry place, etc.). Other components, which could be unstable at the storage conditions for the other components, or which could react slowly with other components, or which is otherwise better kept as a separate stock, may be stored under a different set of conditions (e.g. −20° C. or −80° C., etc.). It is only shortly or immediately before use are these separately stored components brought together to constitute the whole medium. Each separate package may be marketed or sold separately, or as different concentrated stocks (e.g. 2×, 5×, 10×, 100×, 1000×, etc.). In some embodiments, a medium of the instant invention is marketed or sold together with one or more cell lines (e.g., one or more cell line(s) disclosed herein), for whose culture said medium is suitable.

Similarly, the complete medium or individual components, packages thereof could be in the form of dry powder, which, upon reconstitution with an aqueous solution (such as water), will yield the desired medium, or its concentrated stocks (2×, 5×, or 10×, etc.).

Components that can be, or better kept as separate stocks just prior to use include: growth factors (e.g. Epidermal Growth Factor), hormones (e.g. estrogen, progesterone, testosterone), other unstable enzymes/proteins (e.g. transferrin, insulin, cholera toxin, etc.), steroids (e.g. hydrocortisone, cholesterol), vitamins (Vitamins A, Bi₂, K₃), pH indicators (e.g. phenol red), one or more buffer components (e.g. sodium biocarbonate, HEPES) and other chemicals (e.g. glutathione, 17-β-estradiol, O-phosphoryl ethanolamine, etc.).

In certain embodiments, at least some or all components of the medium is in liquid/aqueous form. In other embodiments, at least some or all components of the medium is in solid/powder form.

The media of the invention are suitable for a variety of primary cells from different mammals, including birds, reptiles, human and other non-human mammals. The latter further includes: non-human primates (e.g. monkey, gorilla, etc.), mouse, rat, rabbit, domestic cattle, horse, pig, sheep, goat, dog, and cat.

“Substantially free” as used herein refers to at least about 80% pure, preferably 85%, 90%, 95%, 99% or more pure population of the desired cells in the whole cell population.

The invention also provides an in vitro method of identifying an agent which enhances or positively affects one or more characteristics of intestinal organoids, the characteristics including: differentiation, apoptosis, sensitivity to chemotherapy/radiotherapy, or senescence, the method comprising: (1) contacting a culture of intestinal organoids in the medium of invention with a candidate agent to be assessed for its ability to e.g. enhance or positively affect the one or more characteristics of these organoids, under conditions appropriate for the agent to enter cells; (2) determining the extent to which the characteristics is e.g. enhanced or positively affected in the presence of the candidate agent to be assessed; and, (3) comparing the extent determined with the characteristics of the intestinal organoids under the same conditions, but in the absence of the candidate agent to be assessed, wherein if the characteristics is substantially enhanced or positively affected in the presence of the candidate agent to be assessed than in its absence, the candidate agent to be assessed is an agent which enhances or positively affects one or more the characteristics of the intestinal organoids.

The invention similarly also provides an in vivo method of identifying an agent which inhibits or negatively affects one or more characteristics of the intestinal organoids.

In certain embodiments, the agent is an RNAi molecule. In certain embodiments, the agent is a siRNA molecule. In certain embodiments, the agent is a chemical compound.

In the present invention, “isolated” refers to material removed from its original environment (e.g., the natural environment if it is naturally occurring), and thus is altered “by the hand of man” from its natural state. For example, an isolated polynucleotide could be part of a vector or a composition of matter, or could be contained within a cell, and still be “isolated” because that vector, composition of matter, or particular cell is not the original environment of the polynucleotide. The term “isolated” does not refer to genomic or cDNA libraries, whole cell total or mRNA preparations, genomic DNA preparations (including those separated by electrophoresis and transferred onto blots), sheared whole cell genomic DNA preparations or other compositions where the art demonstrates no distinguishing features of the polynucleotide/sequences of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. However, a nucleic acid contained in a clone that is a member of a library (e.g., a genomic or cDNA library) that has not been isolated from other members of the library (e.g., in the form of a homogeneous solution containing the clone and other members of the library) or a chromosome removed from a cell or a cell lysate (e.g., a “chromosome spread”, as in a karyotype), or a preparation of randomly sheared genomic DNA or a preparation of genomic DNA cut with one or more restriction enzymes is not “isolated” for the purposes of this invention. As discussed further herein, isolated nucleic acid molecules according to the present invention may be produced naturally, recombinantly, or synthetically.

In the present invention, a “secreted” protein refers to a protein capable of being directed to the ER, secretory vesicles, or the extracellular space as a result of a signal sequence, as well as a protein released into the extracellular space without necessarily containing a signal sequence. If the secreted protein is released into the extracellular space, the secreted protein can undergo extracellular processing to produce a “mature” protein. Release into the extracellular space can occur by many mechanisms, including exocytosis and proteolytic cleavage.

“Polynucleotides” can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotides can be composed of triple-stranded regions comprising RNA or DNA or both RNA and DNA. Polynucleotides may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

The expression “polynucleotide encoding a polypeptide” encompasses a polynucleotide which includes only coding sequence for the polypeptide as well as a polynucleotide which includes additional coding and/or non-coding sequence.

“Stringent hybridization conditions” refers to an overnight incubation at 42 degree C. in a solution comprising 50% formamide, 5×SSC (750 mM NaCl, 75 mM trisodium citrate), 50 mM sodium phosphate (pH 7.6), 5×Denhardt's solution, 10% dextran sulfate, and 20 g/ml denatured, sheared salmon sperm DNA, followed by washing the filters in 0.1×SSC at about 50 degree C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37 degree C. in a solution comprising 6×SSPE (20×SSPE=3M NaCl; 0.2M NaH2PO4; 0.02M EDTA, pH 7.4), 0.5% SDS, 30% formamide, 100 g/ml salmon sperm blocking DNA; followed by washes at 50 degree C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

The terms “fragment,” “derivative” and “analog” when referring to polypeptides means polypeptides which either retain substantially the same biological function or activity as such polypeptides. An analog includes a proprotein which can be activated by cleavage of the proprotein portion to produce an active mature polypeptide.

The term “gene” means the segment of DNA involved in producing a polypeptide chain; it includes regions preceding and following the coding region “leader and trailer” as well as intervening sequences (introns) between individual coding segments (exons).

Polypeptides can be composed of amino acids joined to each other by peptide bonds or modified peptide bonds, i.e., peptide isosteres, and may contain amino acids other than the 20 gene-encoded amino acids. The polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in the polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include, but are not limited to, acetylation, acylation, biotinylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, denivatization by known protecting/blocking groups, disulfide bond formation, demethylation, formation of covalent cross-links, formation of cysteine, formation of pyroglutamate, formylation, gamma-carboxylation, glycosylation, GPI anchor formation, hydroxylation, iodination, linkage to an antibody molecule or other cellular ligand, methylation, myristoylation, oxidation, pegylation, proteolytic processing (e.g., cleavage), phosphorylation, prenylation, racemization, selenoylation, sulfation, transfer-RNA mediated addition of amino acids to proteins such as arginylation, and ubiquitination. (See, for instance, PROTEINS-STRUCTURE AND MOLECULAR PROPERTIES, 2nd Ed., T. E. Creighton, W. H. Freeman and Company, New York (1993); POSTTRANSLATIONAL COVALENT MODIFICATION OF PROTEINS, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992).)

A polypeptide fragment “having biological activity” refers to polypeptides exhibiting activity similar, but not necessarily identical to, an activity of the original polypeptide, including mature forms, as measured in a particular biological assay, with or without dose dependency. In the case where dose dependency does exist, it need not be identical to that of the polypeptide, but rather substantially similar to the dose-dependence in a given activity as compared to the original polypeptide (i.e., the candidate polypeptide will exhibit greater activity or not more than about 25-fold less and, in some embodiments, not more than about tenfold less activity, or not more than about three-fold less activity relative to the original polypeptide.)

Species homologs may be isolated and identified by making suitable probes or primers from the sequences provided herein and screening a suitable nucleic acid source for the desired homologue.

“Variant” refers to a polynucleotide or polypeptide differing from the original polynucleotide or polypeptide, but retaining essential properties thereof. Generally, variants are overall closely similar, and, in many regions, identical to the original polynucleotide or polypeptide.

As a practical matter, whether any particular nucleic acid molecule or polypeptide is at least 80%, 85%, 90%, 92%, 95%, 96%, 97%, 98%, 99%, or 100% identical to a nucleotide sequence of the present invention can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Blosci. (1990) 6:237-245). In a sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identity are: Matrix=Unitary, k-tuple=4, Mismatch Penalty-−1, Joining Penalty-−30, Randomization Group Length=0, Cutoff Score=I, Gap Penalty-−5, Gap Size Penalty 0.05, Window Size=500 or the length of the subject nucleotide sequence, whichever is shorter. If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score. For example, a 90 base subject sequence is aligned to a 100 base query sequence to determine percent identity. The deletions occur at the 5′ end of the subject sequence and therefore, the FASTDB alignment does not show a matched/alignment of the first 10 bases at 5′ end. The 10 impaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequence are manually corrected for.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 92%, 95%, 96%, 97%, 98%, 99%, or 100% identical to, for instance, the amino acid sequences shown in a sequence or to the amino acid sequence encoded by deposited DNA clone can be determined conventionally using known computer programs. A preferred method for determining, the best overall match between a query sequence (a sequence of the present invention) and a subject sequence, also referred to as a global sequence alignment, can be determined using the FASTDB computer program based on the algorithm of Brutlag et al. (Comp. App. Biosci. (1990) 6:237-245). In a sequence alignment the query and subject sequences are either both nucleotide sequences or both amino acid sequences. The result of said global sequence alignment is in percent identity. Preferred parameters used in a FASTDB amino acid alignment are: Matrix=PAM 0, k-tuple=2, Mismatch Penalty-−I, Joining Penalty=20, Randomization Group Length=0, Cutoff Score=I, Window Size=sequence length, Gap Penalty-−5, Gap Size Penalty-−0.05, Window Size=500 or the length of the subject amino acid sequence, whichever is shorter. If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence. Only residue positions outside the N- and C-terminal ends of the subject sequence, as displayed in the FASTDB alignment, which are not matched/aligned with the query sequence are manually corrected for. No other manual corrections are to be made for the purposes of the present invention.

Naturally occurring protein variants are called “allelic variants,” and refer to one of several alternate forms of a gene occupying a given locus on a chromosome of an organism. (Genes 11, Lewin, B., ed., John Wiley & Sons, New York (1985).) These allelic variants can vary at either the polynucleotide and/or polypeptide level. Alternatively, non-naturally occurring variants may be produced by mutagenesis techniques or by direct synthesis.

Using known methods of protein engineering and recombinant DNA technology, variants may be generated to improve or alter the characteristics of polypeptides. For instance, one or more amino acids can be deleted from the N-terminus or C-terminus of a secreted protein without substantial loss of biological function. The authors of Ron et al., J. Biol. Chem. 268: 2984-2988 (1993), reported variant KGF proteins having hepanin binding activity even after deleting 3, 8, or 27 amino-terminal amino acid residues. Similarly, Interferon gamma exhibited up to ten times higher activity after deleting 8-10 amino acid residues from the carboxy terminus of this protein (Dobeli et al., J. Biotechnology 7:199-216 (1988)). Moreover, ample evidence demonstrates that variants often retain a biological activity similar to that of the naturally occurring protein. For example, Gayle and co-workers (J. Biol. Chem 268:22105-22111 (1993)) conducted extensive mutational analysis of human cytokine IL-1a. They used random mutagenesis to generate over 3,500 individual IL-1a mutants that averaged 2.5 amino acid changes per variant over the entire length of the molecule. Multiple mutations were examined at every possible amino acid position. The investigators found that “[most of the molecule could be altered with little effect on either [binding or biological activity].” (See, Abstract.) In fact, only 23 unique amino acid sequences, out of more than 3,500 nucleotide sequences examined, produced a protein that significantly differed in activity from wild-type. Furthermore, even if deleting one or more amino acids from the N-terminus or C-terminus of a polypeptide results in modification or loss of one or more biological functions, other biological activities may still be retained. For example, the ability of a deletion variant to induce and/or to bind antibodies which recognize the secreted form will likely be retained when less than the majority of the residues of the secreted form are removed from the N-terminus or C-terminus. Whether a particular polypeptide lacking N- or C-terminal residues of a protein retains such immunogenic activities can readily be determined by routine methods described herein and otherwise known in the art.

By “biological sample” is intended any biological sample obtained from an individual, body fluid, cell line, tissue culture, or other source which contains the polypeptide of the present invention or mRNA. As indicated, biological samples include body fluids (such as semen, lymph, sera, plasma, urine, synovial fluid and spinal fluid), and other tissue sources found to express the polypeptide of the present invention. Methods for obtaining tissue biopsies and body fluids from mammals are well known in the art. Where the biological sample is to include mRNA, a tissue biopsy is the preferred source.

“Matrigel” is the trade name for a gelatinous protein mixture secreted by mouse tumor cells and marketed by BD Biosciences. This mixture resembles the complex extracellular environment found in many tissues and is used by cell biologists as a substrate for cell culture. A common laboratory procedure is to dispense small volumes of chilled (4° C.) Matrigel onto plastic tissue culture labware. When incubated at 37° C. (body temperature) the Matrigel proteins self-assemble producing a thin film that covers the surface of the labware. Cells cultured on Matrigel demonstrate complex cellular behavior that is otherwise impossible to observe under laboratory conditions. For example, endothelial cells create intricate spiderweb-like networks on Matrigel coated surfaces but not on plastic surfaces. Such networks are highly suggestive of the microvascular capillary systems that suffuse living tissues with blood. Hence, the process by which endothelial cells construct such networks is of great interest to biological researchers and Matrigel allows them to observe this. In some instances, researchers use greater volumes of Matrigel to produce thick three-dimensional gels. The utility of thick gels is that they induce cells to migrate from the surface to the interior of the gel. This migratory behavior is studied by researchers as a model of tumor cell metastasis. Pharmaceutical scientists use Matrigel to screen drug molecules. A typical experiment consists of adding a test molecule to Matrigel and observing cellular behavior. Test molecules that promote endothelial cell network formation are candidates for tissue regeneration therapies whereas test molecules that inhibit endothelial cell network formation are candidates for anti-cancer therapies. Likewise, test molecules that inhibit tumor cell migration may also have potential as anti-cancer drugs. The ability of Matrigel to stimulate complex cell behavior is a consequence of its heterogeneous composition. The chief components of Matrigel are structural proteins such as laminin and collagen which present cultured cells with the adhesive peptide sequences that they would encounter in their natural environment. Also present are growth factors that promote differentiation and proliferation of many cell types. Matrigel contains numerous other proteins in small amounts and its exact composition is unknown. Matrigel is also used as an attachment substrate in embryonic stem cell culture. When embryonic stem cells are grown in the absence of feeder cells, extracellular matrix components such as Matrigel are necessary to maintain the pluripotent, undifferentiated state (self-renewal).

Neuregulin 1, also known as NRG1, ARIA, GGF, GGF2, HGL, HRG, HRG1, HRGA, MST131, MSTP131, NDF, NRG1-IT2, or SMDF, is a member of the epidermal growth factor family that in humans is encoded by the NRG1 gene. NRG1 is one of four proteins in the neuregulin family that act on the EGFR family of receptors. Neuregulin 1 is produced in numerous isoforms by alternative splicing, which allows it to perform a wide variety of functions. A suitable NRG1 is the one sold by R&D Systems (Cat #5898-NR-050; NS0-derived human Neuregulin-1/NRG1 protein, Ser20-Lys241, with a C-terminal 6-His tag).

All-Trans Retinoic Acid, also known as atRA, NSC 122758, Retinoic acid, Trans retinoic acid, Tretinoin, and Vitamin A acid, is a derivative of Vitamin A that functions as a ligand for the retinoic acid receptor (RAR, IC₅₀=14 nM). RARs heterodimerize with retinoid X receptors (RXRs) and bind to retinoic acid response elements (RAREs) in DNA and act as transcription factors, altering gene expression.

R-spondin-1, also known as RSPO1, CRISTIN3, and RSPO, is a secreted protein that in humans is encoded by the Rspo1 gene, found on chromosome 1. In humans, it interacts with WNT4 in the process of female sex development. Loss of function can cause female to male sex reversal. Furthermore, it promotes canonical WNT/s catenin signaling.

Noggin, also known as NOG, Nog, SYM1, SYNS1, and SYNS1A, is a protein that is involved in the development of many body tissues, including nerve tissue, muscles, and bones. In humans, noggin is encoded by the NOG gene. The amino acid sequence of human noggin is highly homologous to that of rat, mouse, and Xenopus. Noggin is an inhibitor of several bone morphogenetic proteins (BMPs): it inhibits at least BMP2, BMP4, BMP5, BMP6, BMP7, BMP13, and BMP14.

Insulin-like growth factor 1 (IGF-1), also called somatomedin C, IGF-I, IGF1A, IGFI, and MGF, is a hormone similar in molecular structure to insulin which plays an important role in childhood growth, and has anabolic effects in adults. IGF-1 is a protein that in humans is encoded by the IGF1 gene. IGF-1 consists of 70 amino acids in a single chain with three intramolecular disulfide bridges. IGF-1 has a molecular weight of 7,649 Daltons. IGF-1 is produced primarily by the liver. Production is stimulated by growth hormone (GH). Most of IGF-1 is bound to one of 6 binding proteins (IGF-BP). IGFBP-1 is regulated by insulin.

FGF2, also known as BFGF, FGF-2, FGFB, HBGF-2, fibroblast growth factor 2, basic fibroblast growth factor (bFGF) and FGF-β, is a growth factor and signaling protein encoded by the FGF2 gene. It binds to and exerts effects via specific fibroblast growth factor receptor (FGFR) proteins, themselves a family of closely related molecules.

Gastrin, also known as GAST, and GAS, is a peptide hormone that stimulates secretion of gastric acid (HCl) by the parietal cells of the stomach and aids in gastric motility. It is released by G cells in the pyloric antrum of the stomach, duodenum, and the pancreas. Gastrin binds to cholecystokinin B receptors to stimulate the release of histamines in enterochromaffin-like cells, and it induces the insertion of K+/H+ ATPase pumps into the apical membrane of parietal cells (which in turn increases H+ release into the stomach cavity). Its release is stimulated by peptides in the lumen of the stomach.

The WNT gene family consists of structurally related genes that encode secreted signaling proteins. These proteins have been implicated in oncogenesis and in several developmental processes, including regulation of cell fate and patterning during embryogenesis. This gene is a member of the WNT gene family. It is very conserved in evolution, and the protein encoded by this gene is known to be 98% identical to the mouse Wnt1 protein at the amino acid level.

Miao et al. (Volume 27, Issue 5, 5 Nov. 2020, Pages 840-851.e6) designed and engineered Wnt NGS. Wnt NGS is a water-soluble, Fzd subtype-specific “next-generation surrogate” (NGS) Wnts that hetero-dimerize Fzd and Lrp6. NGS Wnt supports long-term expansion of multiple different types of organoids, including kidney, colon, hepatocyte, ovarian, and breast. NGS Wnts are superior to Wnt3a (another possible alternative of the media of the invention) conditioned media in organoid expansion and single-cell organoid outgrowth.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting. Moreover, it is to be noted that despite that fact that the above description concentrates on medical uses, the methods and agents of the invention are also suitable for any non-medical use.

### EXAMPLES

Example protocols for the preparation of different media for the culture of intestinal organoids:

### Small Intestine Medium Regime 1× Formulation

### First Culture Medium for the First e.g. 4 Days of Culture

0.5 nM Wnt-NGS
1 μg/ml Recombinant R-Spondin-1
100 ng/ml Recombinant Noggin
1× B27™ supplement
1.25 mM NAC
50 ng/ml hEGF
1 ng/ml hNRG1
0.5 μM A83-01
100 ng/ml hIGF1
50 ng/ml hFGF2
25 nM Gastrin
10 μM ROCK-Inhibitor (Y-27632)
in DMEM/F12+15 mM HEPES, 1× GlutaMax (1:100 #35050-038), and 100 μg/ml PenStrep (#15140-122)
   Second Medium after e.g. 4 Days of Culture
0.5 μg/ml Recombinant R-Spondin1
100 ng/ml Recombinant Noggin
1× B27™ supplement
1.25 mM NAC
5 ng/ml hEGF
10 ng/ml hNRG1
100 ng/ml hIGF1
50 ng/ml hFGF2
25 nM Gastrin
2.5 μM atRA (all-trans Retonic Acid)
in DMEM/F12+15 mM HEPES, 1× GlutaMax (1:100 #35050-038), and 100 μg/ml PenStrep (#15140-122)

### Colon Medium Regime 1× Formulation

### First Culture Medium for the First e.g. 4 Days of Culture

0.5 nM Wnt (NGS)
1 μg/ml Recombinant R-Spondin1
100 ng/ml Recombinant Noggin
1× B27™ supplement
1.25 mM
50 ng/ml hEGF
1 ng/ml hNRG1
0.5 μM A83-01
100 ng/ml hIGF1
50 ng/ml hFGF2
25 nM Gastrin
10 μM ROCK-Inhibitor (Y-27632)
in DMEM/F12+15 mM HEPES, 1× GlutaMax (1:100 #35050-038), and 100 μg/ml PenStrep (#15140-122)
   Second Medium after e.g. 4 Days of Culture
0.1 nM Wnt (NGS)
0.5 μg/ml Recombinant R-Spondin1
100 ng/ml Recombinant Noggin
1× B27™ supplement
1.25 mM NAC
5 ng/ml hEGF
10 ng/ml hNRG1
100 ng/ml hIGF1
50 ng/ml hFGF2
25 nM Gastrin 25 nM
0.1 μM atRA
in DMEM/F12+15 mM HEPES, 1× GlutaMax (1:100 #35050-038), and 100 μg/ml PenStrep (#15140-122)

### Material and Methods

### Human Organoid Lines

All patients gave informed consent for extra biopsy samples to be taken for research use when undergoing elective colonoscopy. Human healthy colon (W18-50157; HUB-02-A2-040) and small intestinal (D1n (HUB-04-A2-001)) organoids were provided by our collaborator HUB Organoids in Utrecht, the Netherlands.

### Mouse Organoid Lines

All animal experiments were approved by the Basel Cantonal Veterinary Authorities and conducted in accordance with the Guide for Care and Use of Laboratory Animals. Male and female outbred mice between 7 and 15 weeks old were used for all experiments. Mouse lines used: C57BL/6 wild type (Charles River Laboratories) and Lgr5::DTR-EGFP (Genentech, de Sauvage laboratory).

### Human Organoid Culture

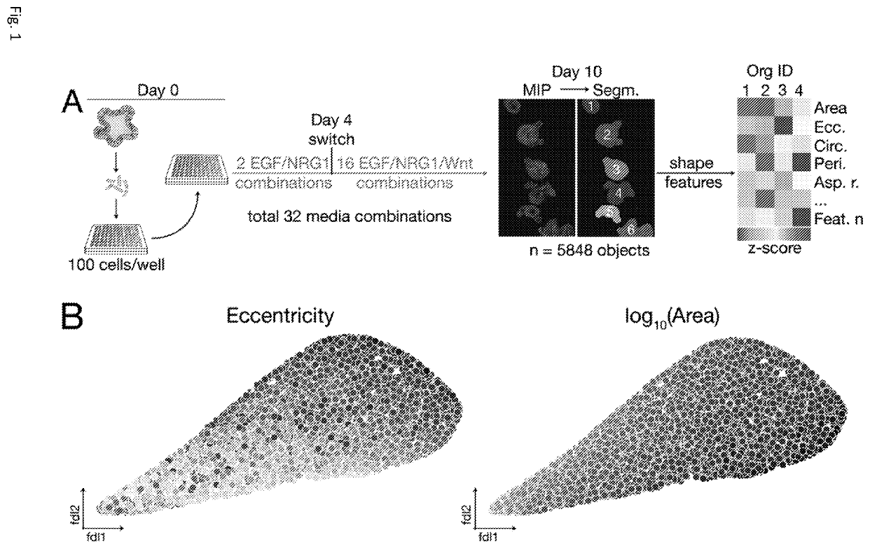

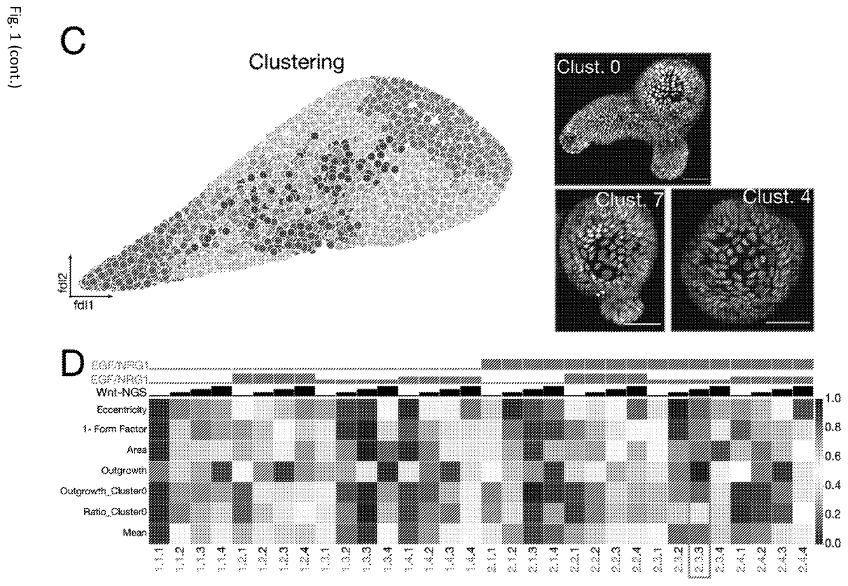

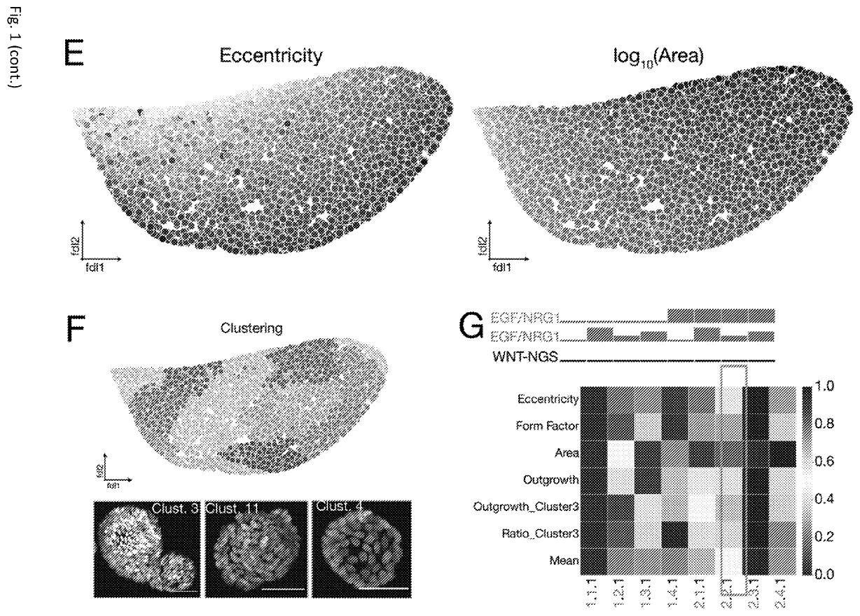

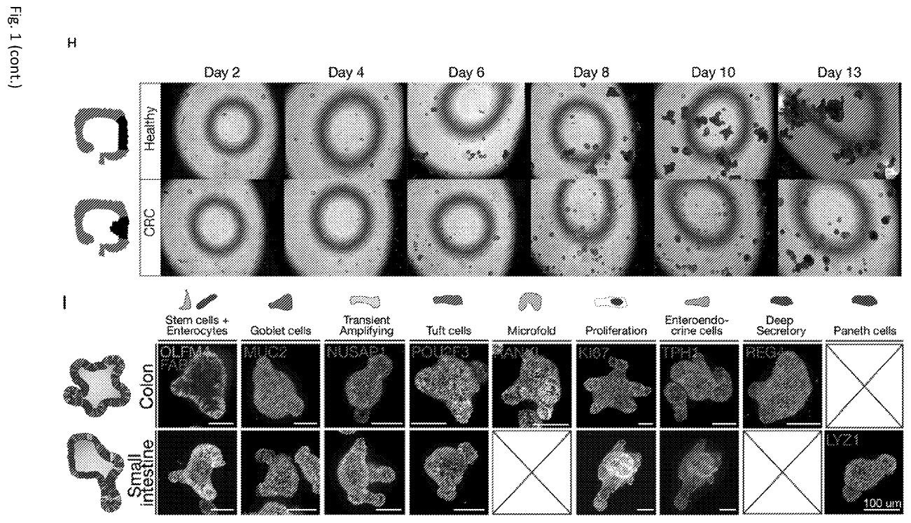

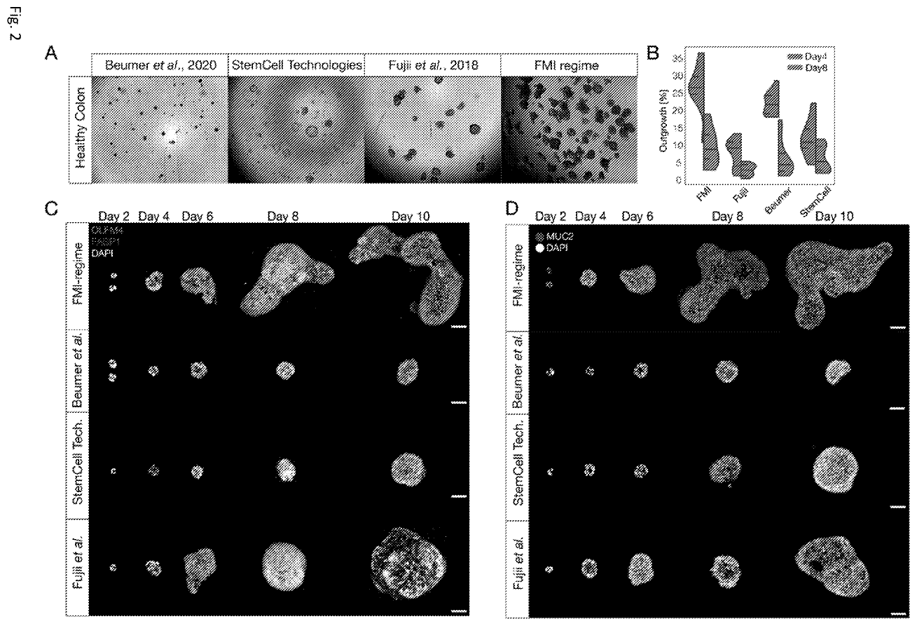

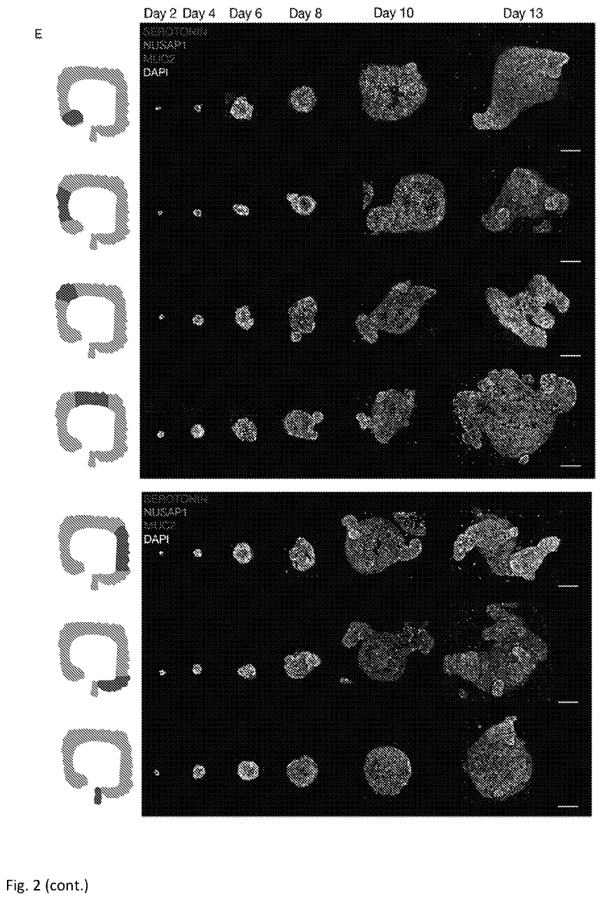

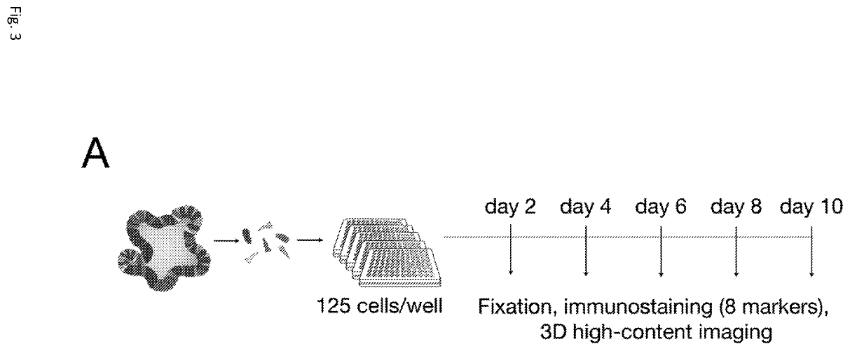

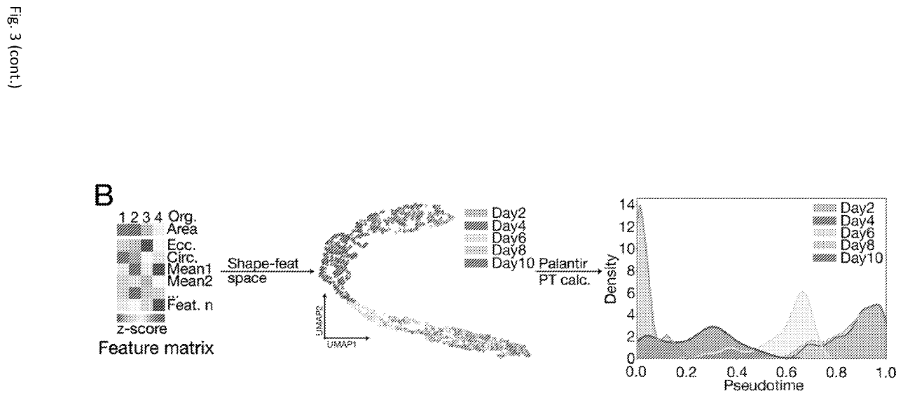

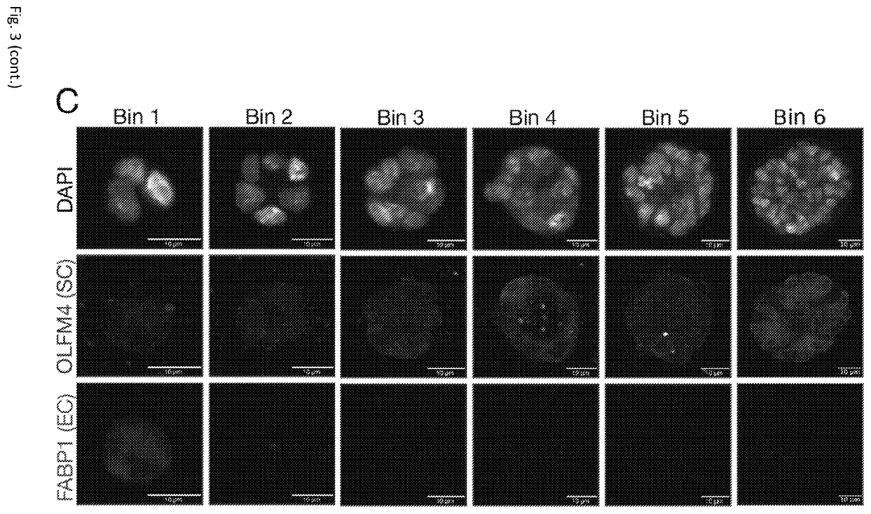

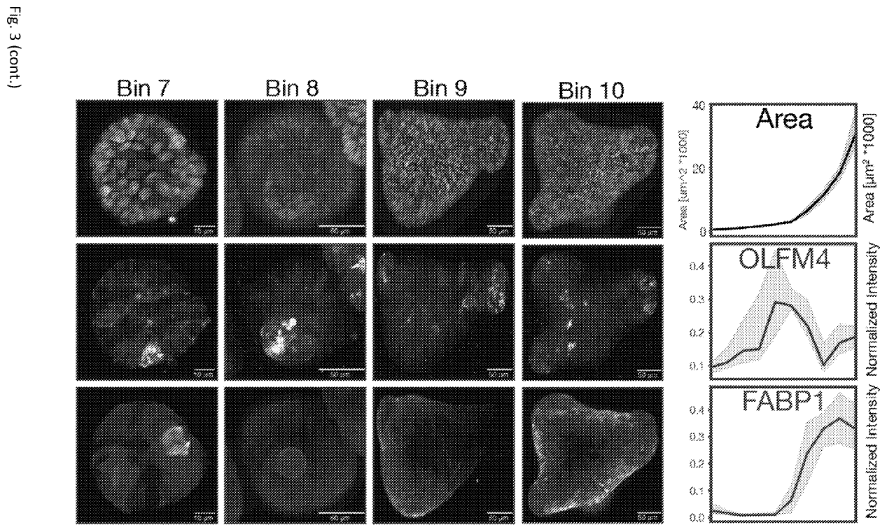

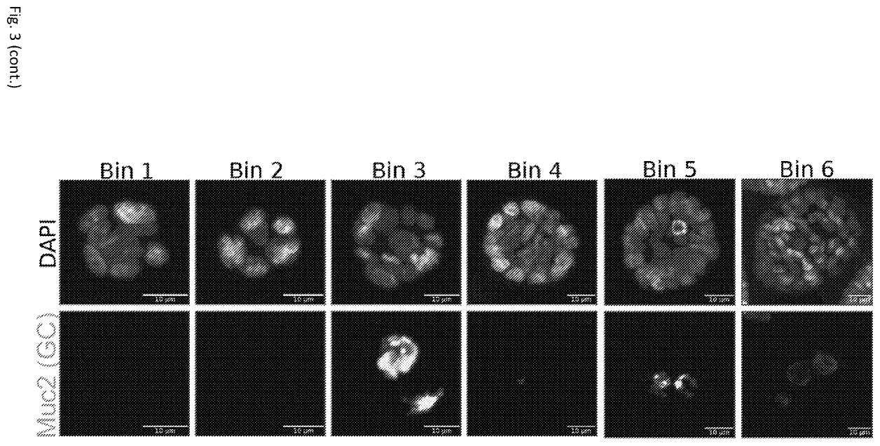

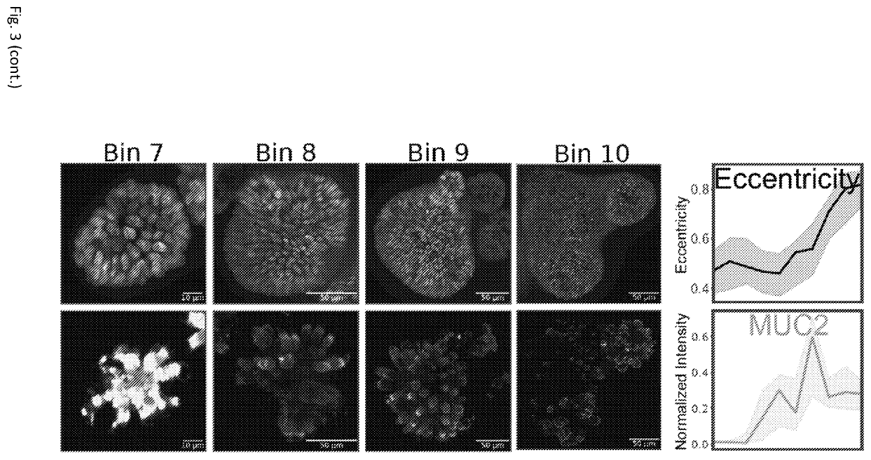

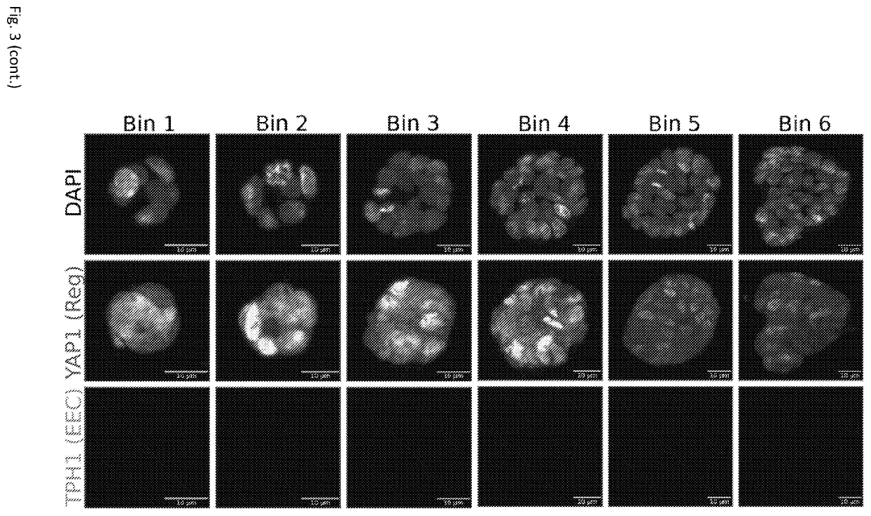

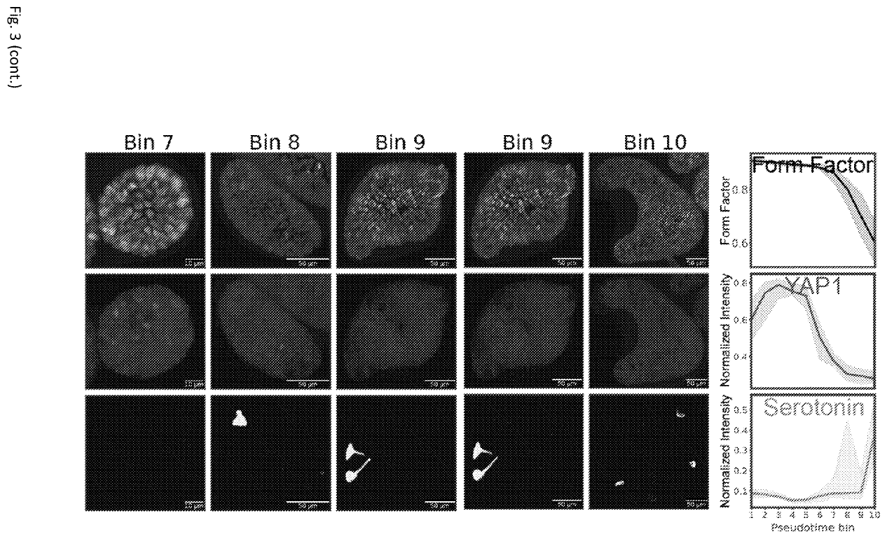

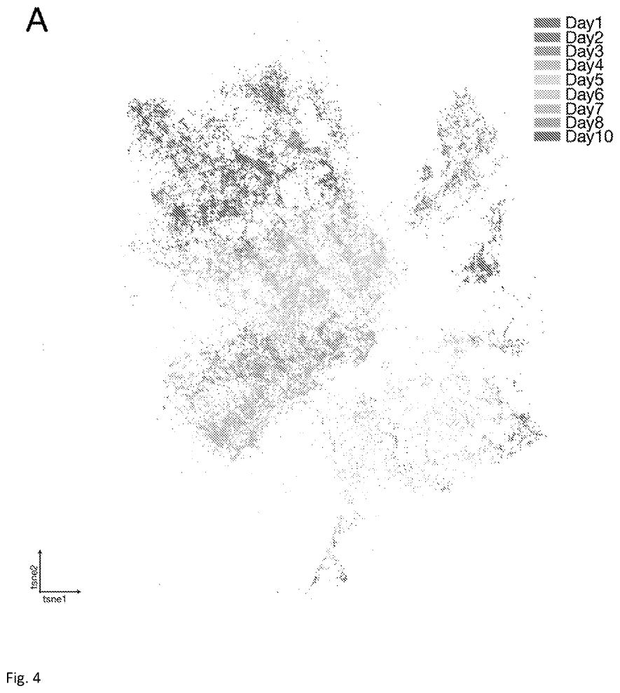

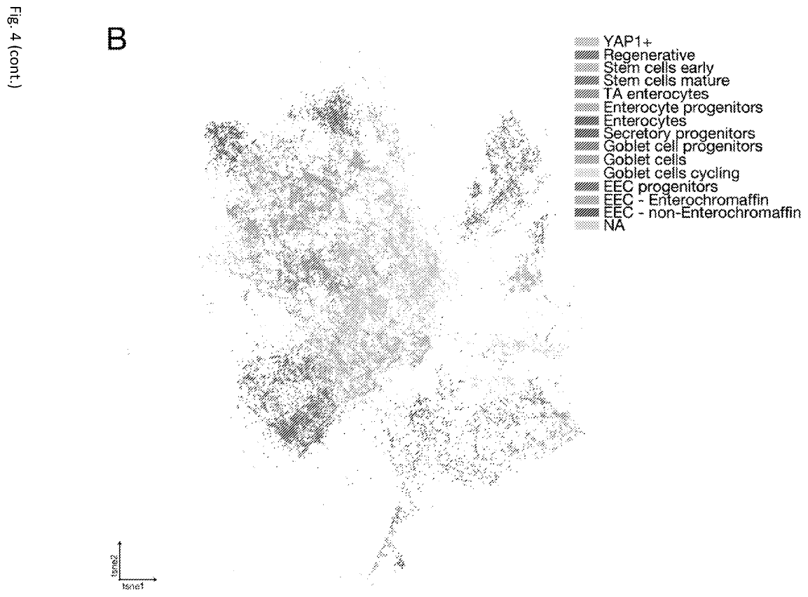

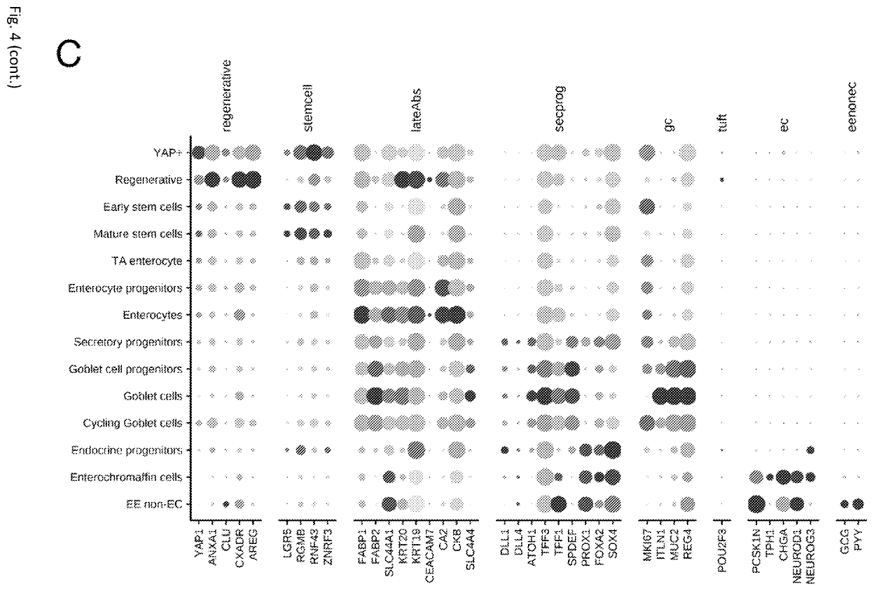

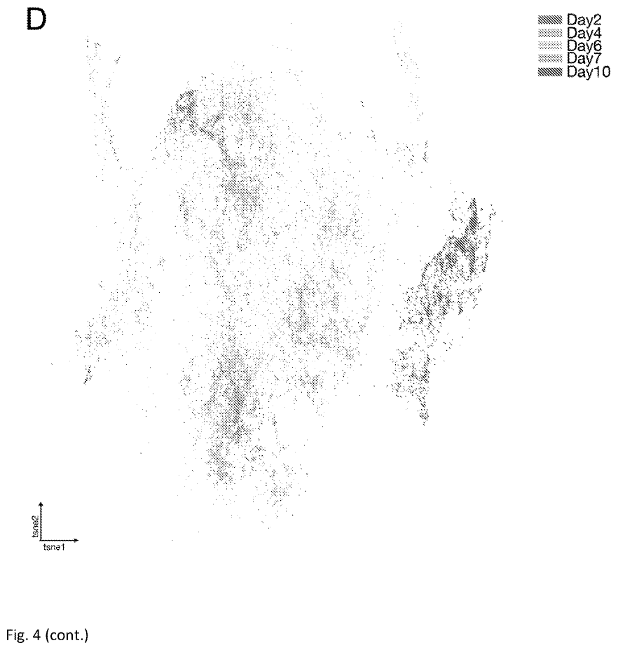

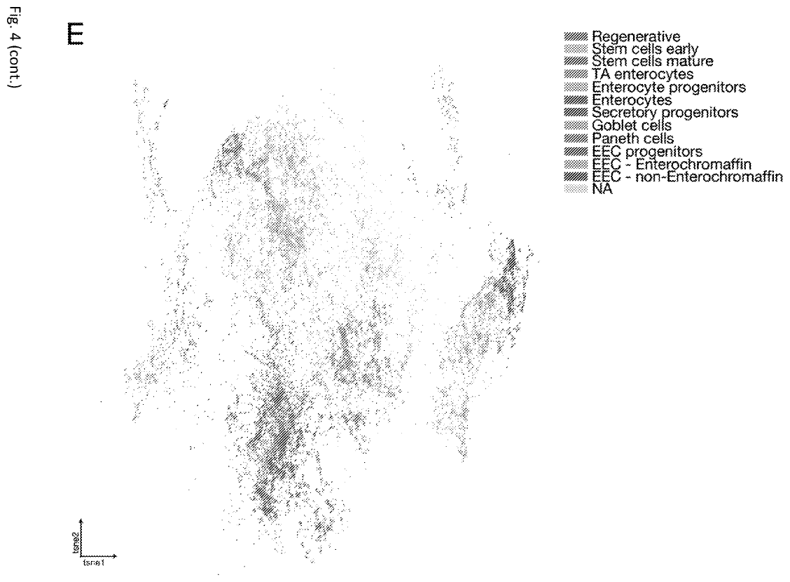

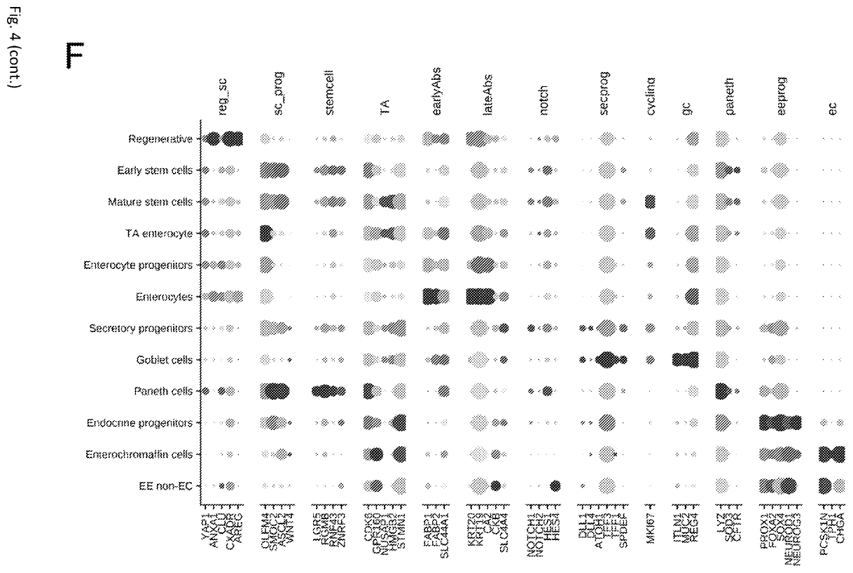

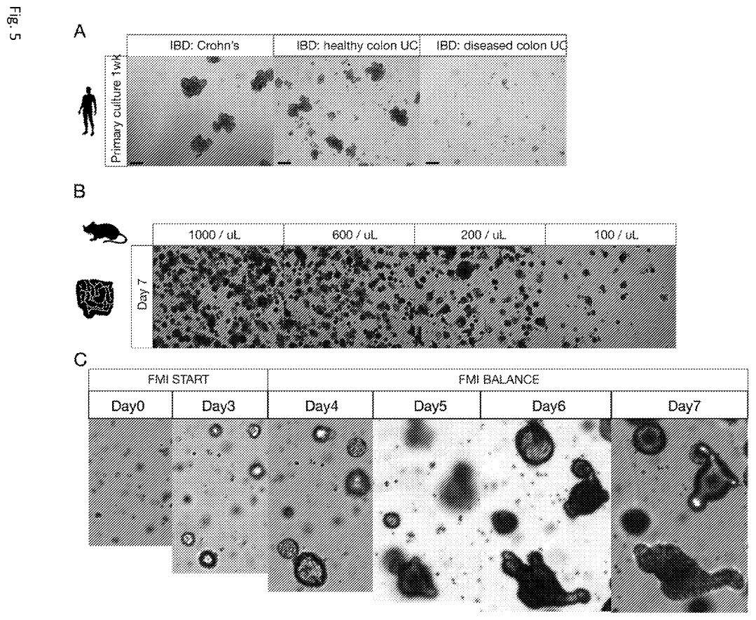

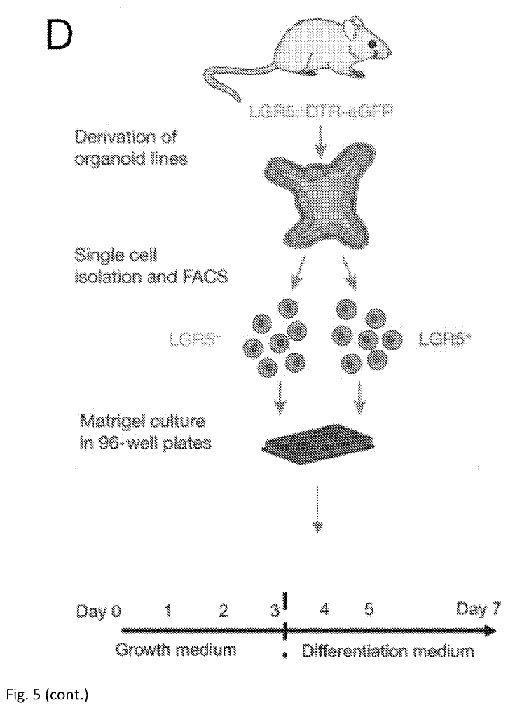

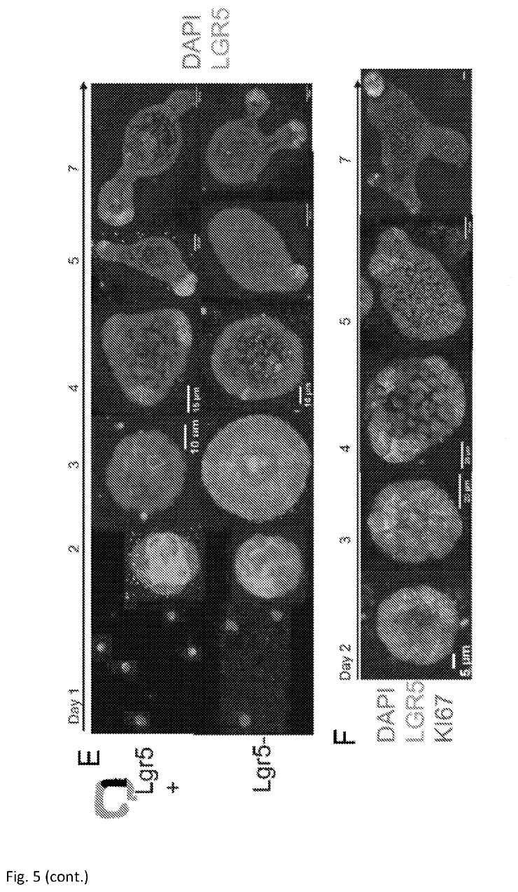

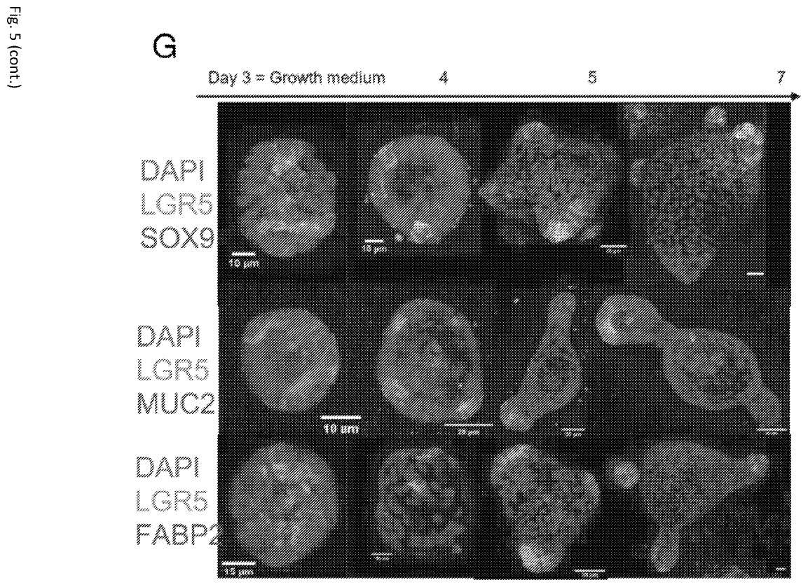

Organoids grown from primary tissue were generated from isolated crypts of a human biopsy collected by a colonoscopy as previous described¹. Organoids were kept in FMI-START medium (advanced DMEM/F-12 with 15 mM HEPES (STEMCELL Technologies) supplemented with 100 μg/ml Penicillin-Streptomycin, 1× Glutamax (Thermo Fisher Scientific), 1× B27 (Thermo Fisher Scientific), 1.25 mM N-acetylcysteine (Sigma), 1 μg/ml recombinant human R-Spondin-1 (kind gift from Novartis), 100 ng/ml Noggin (PeproTech), 0.5 nM WNT-NGS (U-Protein Express), 0.5 μM A83-01 (Tocris), 100 ng/ml human IGF1 (R&D Systems), 50 ng/ml human FGF2 (R&D systems), 25 nM Gastrin (Sigma), 1 ng/ml human NRG1 (R&D Systems), and 50 ng/ml human or murine EGF (Thermo Scientific)) supplemented with 10 mM Y27632 (Rock inhibitor, Stem Cell technologies) for 4 days after trypsinization. After 4 days organoids were overlayed with either FMI-BALANCE colon (advanced DMEM/F-12 with 15 mM HEPES (STEMCELL Technologies) supplemented with 100 μg/ml Penicillin-Streptomycin, 1× Glutamax (Thermo Fisher Scientific), 1×B27 (Thermo Fisher Scientific), 1.25 mM N-acetylcysteine (Sigma), 0.5 μg/ml recombinant human R-Spondin-1 (kind gift from Novartis), 100 ng/ml Noggin (PeproTech), 0.1 nM WNT-NGS (U-Protein Express), 100 ng/ml human IGF1 (R&D Systems), 50 ng/ml human FGF2 (R&D systems), 25 nM Gastrin (Sigma), 10 ng/ml human NRG1 (R&D Systems), 10 μM (FIG. 1A-G)/0.1 μM (else) atRA (Sigma-Aldrich), and 5 ng/ml human or murine EGF (Thermo Scientific)) or FMI-BALANCE small intestine (advanced DMEM/F-12 with 15 mM HEPES (STEMCELL Technologies) supplemented with 100 μg/ml Penicillin-Streptomycin, 1× Glutamax (Thermo Fisher Scientific), 1×B27 (Thermo Fisher Scientific), 1.25 mM N-acetylcysteine (Sigma), 0.5 μg/ml recombinant human R-Spondin-1 (kind gift from Novartis), 100 ng/ml Noggin (PeproTech), 100 ng/ml human IGF1 (R&D Systems), 50 ng/ml human FGF2 (R&D systems), 25 nM Gastrin (Sigma), 10 ng/ml human NRG1 (R&D Systems), 10 μM (FIG. 1A-G)/1 μM (FIG. 11)/2.5 μM (else) atRA (Sigma-Aldrich), and 5 ng/ml human or murine EGF (Thermo Scientific)) depending on their origin. The medium was replaced at day 7 and once more at day 10 for organoids of small intestinal origin due to prolonged maturation times.

### Mouse Organoid Culture

Mouse organoids were generated from isolated crypts of the murine small intestine as previously described². Organoids were kept in FMI-START medium supplemented with 10 μM Y27632 (ROCK inhibitor, STEMCELL Technologies) for 3 days after trypsinization. After 3 days organoids were kept either in FMI-BALANCE colon or FMI-BALANCE small intestine and the medium was replaced once more at day 5.

### Image-Based Screening Assays

Mouse small intestine and colon organoids were collected 5-7 days after passaging and digested with (0.05%) Trypsin EDTA (Gibco) for 5 min at 37° C. Human small intestine and colon organoids were collected 10-13 days after passaging and digested with (0.05%) Trypsin EDTA (Gibco) for 5-10 min at 37° C. Dissociated cells were passed through a cell strainer with a pore size of 40 μm. For indicated experiments, single alive cells were sorted by FACS (Becton Dickinson FACSAria or SONY MA900 cell sorter). Forward scatter and side scatter properties were used to remove cell doublets and dead cells. Dead cells were also filtered away by a DRAQ7 stain (1.5 μM, Thermo Fisher Scientific). Mouse organoid lines were derived from C57BL/6 wild type mice unless indicated otherwise.

Imaging-based experiments where either conducted in 384-well plates (CellCarrier-384, PerkinElmer, cat. nr. 6007550) or 96-well plates (μClear, Greiner, cat. nr. 655090).

For experiments in 384-well plates, cells were resuspended in FMI-START containing 10 μM Y27632 (ROCK inhibitor, STEMCELL Technologies) to reach a density of ˜6-50 cells/μl, depending on the experiment and line used. Next, wells of a 384 well plate were covered with 10 μl/well of an ice-cold 2:1 Matrigel (Corning): FMI-START mixture using the Assist Plus pipetting robot (Integra) under constant cooling, covering the entire bottom of the well. Plates were centrifuged to reach a flat layer of the Matrigel mixture before being incubated for 10 min at RT to allow for slight solidification. After that, 40 μl of the cell mixture containing 10 μM Y27632 (ROCK inhibitor, STEMCELL Technologies) was overlayed carefully onto prepared wells. Cells were centrifuged into the Matrigel carefully using a slow speed (50-70 rcf for 5 s) centrifuge. Finally, plates were placed into the incubator (37° C., 5% CO₂) for culturing.

For experiments in 96-well plates, cells were resuspended in an ice-cold 2:1 Matrigel (Corning): FMI-START mixture containing 10 μM Y27632 (ROCK inhibitor, STEMCELL Technologies) to reach a density of ˜6-50 cells/μl (depending on the experiment and line used) and seeded in 5 μl drops into the center of wells. Plates were incubated at 37° C. for 20 minutes to allow solidification of the Matrigel. After this time, 100 μl of FMI-START supplemented with 10 μM Y27632 (ROCK inhibitor, STEMCELL Technologies) was added for culturing in an incubator (37° C., 5% CO₂).

For testing the different media regimes, previously described^{1,3}*, it was decided to change media at the same time as for the FMI-medium regime at day 4 and 7. In case of the Fujii et al. medium regime³ there was no differentiation medium required compared to the Beumer et al.¹ and Stem Cell Technologies* regimes. Therefore, the inventors cultured the organoids in the Fujii et al. regime constantly with the same medium with refreshments at day 4 and 7.

During the experiment, medium was changed to FMI-BALANCE on day 4 and refreshed once at day 7. Small intestinal organoids received an additional medium refresh at day 10 to allow growth until maturation at day 13.

For the mouse organoids cells were mixed with Matrigel (Corning) in a medium to Matrigel ratio of 1:1. In each well of a 96 well plate (μClear, Greiner, cat. nr. 655090), 5 μl droplets with different densities were seeded. After 20 min of solidification at 37° C., 100 μl of medium was overlaid.

### Fixed Sample Preparation and Imaging

To allow imaging of all organoids within a similar z-range, each well plate was centrifuged at 1000 rcf for 10 min in a pre-cooled centrifuge at 10° C. prior to fixation. Organoids were fixed at indicated time points in 4% PFA (Electron Microscopy Sciences) in PBS for 30 min at room temperature.

For the image-based screening assays, organoids were permeabilized and blocked with 0.5% Triton X-100 (Sigma-Aldrich), 3% donkey serum (Sigma-Aldrich), 100 mM NH4CI in PBS for 3 h (human) or 1 h (mouse) at RT. Primary and secondary antibodies were diluted in antibody buffer (0.1% Triton X-100, 3% donkey serum in PBS) and applied as described in Table 2. For staining of nuclei, 0.2 μg/ml DAPI (4′,6-Diamidino-2-Phenylindole, Invitrogen) was added to the secondary antibody staining mix. Fixation, blocking/permeabilization, and washing steps were performed using EL406 Combination Washer Dispenser (BioTek Instruments).

High-throughput imaging was done with one of two automated spinning disk microscopes (Molecular Devices ImageXpress Micro Confocal or Yokogawa CellVoyager 7000S). The Molecular Devices ImageXpress Micro Confocal in combination with a Nikon 20× Plan Apo 0.75 NA objective were used for imaging. For each site, z-planes spanning the organoid-size were acquired. 5 μm z-steps were used in all experiments unless indicated otherwise.

Alternatively, the CellVoyager 7000S in combination with an Olympus 20×UPLSAPO 20×0.75 NA objective was also used for imaging. Here, a search first imaging approach was used (Search First module of Wako Software Suite). For this, in each well one field was acquired with 4× resolution in order to cover the complete well area. This overview was then used to segment organoids on the fly with a custom written ImageJ macro which outputs coordinates of organoid positions. These coordinates were used to generate map of locations for high resolution re-imaging (20×, NA=0.75). For each site, z-planes spanning the organoid-size were acquired. 5 μm z-steps were used in all experiments unless indicated otherwise.

### Image Analysis

Individual acquired field-of-views were first combined to generate well-overviews of every well. Next, maximum intensity projections were generated for all overviews and channels. The DAPI-channel was subsequently used to segment individual organoids using a trained RDCNet Network⁴. After segmentation, areas of single organoids were extracted from the overview based on the segmentation.

All excised areas went through a filtering step to remove wrongly segmented organoids as well as debris based on size, DAPI-brightness or axis ratio. Additionally, organoids which were cut by the imaging-border were excluded for further analysis. Features of remaining organoids were computed primarily using the regionprops functions from the python package scikit-image. Furthermore, outliers were removed by a quantile filter with 0.01 and 0.99 as the lower and upper limits, respectively.

For indicated experiments pseudotime was calculated using the shape-feature space. In short, a subset of shape-features was hand-picked to model organoid maturation through time. This subset was then used to calculate the pseudotime of all organoids using the palantir python package⁵. For this, random organoids were picked of the earliest and latest timepoint to build a trajectory between those using 500 intermediate random waypoints. This process was repeated five times and the average pseudotime per organoid was calculated.

Single Cell RNA Sequencing (scRNAseq) and Raw Data Processing

Single cells were isolated from organoids at indicated time points, passed through a cell strainer with a pore size of 40 μm and used for FACS sorting to discard debris and dead cells. Cellular suspensions were loaded on a 10× Genomics Chromium Single Cell instrument to generate single cell GEMs. Single cell RNA-Seq libraries were prepared using GemCode Single Cell 3′ Gel Bead and Library Kit according to CG00052_SingleCell3′ReagentKitv2UserGuide_RevB. GEMRT was performed in a Bio-Rad PTC-200 Thermal Cycler with semi-skirted 96-Well Plate (Eppendorf P/N 0030 128.605): 53° C. for 45 minutes, 85° C. for 5 minutes; held at 4° C. After RT, GEMs were broken and the single strand cDNA was cleaned up with DynaBeads® MyOne™ Silane Beads (Life Technologies P/N 37002D). cDNA was amplified using a Bio-Rad PTC-200 Thermal cycler with 0.2 ml 8-strip nonFlex PCR tubes, with flat Caps (STARLAB P/N I1402-3700): 98° C. for 3 min; cycled 12×: 98° C. for 15 s, 67° C. for 20 s, and 72° C. for 1 min; 72° C. for 1 min; held at 4° C. Amplified cDNA product was cleaned up with the SPRIselect Reagent Kit (0.6×SPRI). Indexed sequencing libraries were constructed using the reagents in the Chromium Single Cell 3′ library kit V2 (10× Genomics P/N120237), following these steps: 1) Fragmentation, End Repair and A-Tailing; 2) Post Fragmentation, End Repair & A-Tailing Double Sided Size Selection with SPRIselect 10 Reagent Kit (0.6×SPRI and 0.8×SPRI); 3) adaptor ligation; 4) post-ligation cleanups with SPRIselect (0.8×SPRI); 5) sample index PCR using the Chromium Multiplex kit (10× Genomics P/N-120262); 6) Post Sample Index Double Sided Size Selection—with SPRIselect Reagent Kit (0.6×SPRI and 0.8×SPRI). The barcode sequencing libraries were quantified using a Qubit 2.0 with a Qubit™ dsDNA HS Assay Kit (Invitrogen P/N Q32854) and the quality of the libraries were performed on a 2100 Bioanalyzer from Agilent using an Agilent High Sensitivity DNA kit (Agilent P/N 5067-4626). Sequencing libraries were loaded at 10 pM on an Illumina HiSeq2500 with 2×50 paired-end kits using the following read length: 26 cycles Read1, 8 cycles i7 Index and 98 cycles Read2. The CellRanger suite (1.3.0) was used to generate the aggregated gene expression matrix from the BCL files generated by the sequencer based on the mm10 Cell Ranger human genome annotation files.

scRNA Sequencing Analysis

To exclude low-quality cells, the inventors removed cells with less than 4,500 reads (4,000 for the small-intestine dataset) or cells with more than 26% of the transcripts coming from mitochondrial genes. To exclude low-quality features, they removed transcripts that were detected in less than 27 cells. After removing unwanted cells and features from the dataset, they normalized the data by the total expression, multiply it for the mean value of the library size distribution and log-transformed the result. The inventors further excluded from the dataset feature types that poorly contributed to the total UMI count (including non-coding RNA, pseudogenes, ribosomal proteins, snoRNA, and snRNA). After pre-processing our dataset counted 18,469 transcripts in 53,640 cells, sampled across nine timepoints for colon organoids, and 17,195 transcripts in 26,453 cells, sampled across five timepoints for small intestinal organoids.

The inventors identified variable genes using modelGeneVar and getTopHVGs functions from the scran package in Bioconductor, blocking for timepoints and using a threshold of 0.05 for false discovery rates.

The inventors performed a density equalizing principal component analysis, using sketchR, an R implementation of the geometric sketching proposed by Hie et al. 2019.⁶

To visualize the data, they further reduced the dimensionality of the datasets to project the cells in 2D space using T-distributed stochastic neighbor embedding (t-SNE), on the basis of the first 50 principal components. The inventors then used the Seurat R package to perform graph-based clustering. To visualize clusters' expression of known cell type markers, They used the DotPlot function from the Seurat package. They assigned clusters to known cell types based on their relative gene expression. *https://www.stemcell.com/products/intesticult-organoid-differentiation-medium-human.html #section-product-documents

### SUPPLEMENTARY REFERENCES

1. Beumer, J. et al. High-Resolution mRNA and Secretome Atlas of Human Enteroendocrine Cells. Cell 181, 1291-1306.e19 (2020).
2. Sato, T. et al. Single Lgr5 stem cells build crypt-villus structures in vitro without a mesenchymal niche. Nature 459, 262-265 (2009).
3. Fujic, M. et al. Human Intestinal Organoids Maintain Self-Renewal Capacity and Cellular Diversity in Niche-Inspired Culture Condition. Cell Stem Cell 23, 787-793.e6 (2018).
4. Ortiz, R., De Medeiros, G., Peters, A. H. F. M., Liberali, P. & Rempfler, M. RDCNet: Instance segmentation with a minimalist recurrent residual network.
5. Setty, M. et al. Characterization of cell fate probabilities in single-cell data with Palantir. Nat. Biotechnol. 37, 451-460 (2019).
6. Hie, B., Cho, H., DeMeo, B., Bryson, B. & Berger, B. Geometric Sketching Compactly Summarizes the Single-Cell Transcriptomic Landscape. Cell Syst. 8, 483-493.e7 (2019).

TABLE 1

| | | conc. EGF | conc. hNRG1 | conc. EGF | conc. hNRG1 | conc. Wnt-NGS |
|---|---|---|---|---|---|---|
| | Medium | [ng/ml] FMI- | [ng/ml] FMI- | [ng/ml] FMI- | [ng/ml] FMI- | [nM] FMI- |
| Region | key | START | START | BALANCE | BALANCE | BALANCE |
| hColon | 1.1.1 | 5 | 10 | 0 | 10 | 0 |
| (FIG. 1D) | | | | | | |
| hColon | 1.1.2 | 5 | 10 | 0 | 10 | 0.05 |
| (FIG. 1D) | | | | | | |
| hColon | 1.1.3 | 5 | 10 | 0 | 10 | 0.1 |
| (FIG. 1D) | | | | | | |
| hColon | 1.1.4 | 5 | 10 | 0 | 10 | 0.25 |
| (FIG. 1D) | | | | | | |
| hColon | 1.2.1 | 5 | 10 | 50 | 0 | 0 |
| (FIG. 1D) | | | | | | |
| hColon | 1.2.2 | 5 | 10 | 50 | 0 | 0.05 |
| (FIG. 1D) | | | | | | |
| hColon | 1.2.3 | 5 | 10 | 50 | 0 | 0.1 |
| (FIG. 1D) | | | | | | |
| hColon | 1.2.4 | 5 | 10 | 50 | 0 | 0.25 |
| (FIG. 1D) | | | | | | |
| hColon | 1.3.1 | 5 | 10 | 5 | 10 | 0 |
| (FIG. 1D) | | | | | | |
| hColon | 1.3.2 | 5 | 10 | 5 | 10 | 0.05 |
| (FIG. 1D) | | | | | | |
| hColon | 1.3.3 | 5 | 10 | 5 | 10 | 0.1 |
| (FIG. 1D) | | | | | | |
| hColon | 1.3.4 | 5 | 10 | 5 | 10 | 0.25 |
| (FIG. 1D) | | | | | | |
| hColon | 1.4.1 | 5 | 10 | 50 | 1 | 0 |
| (FIG. 1D) | | | | | | |
| hColon | 1.4.2 | 5 | 10 | 50 | 1 | 0.05 |
| (FIG. 1D) | | | | | | |
| hColon | 1.4.3 | 5 | 10 | 50 | 1 | 0.1 |
| (FIG. 1D) | | | | | | |
| hColon | 1.4.4 | 5 | 10 | 50 | 1 | 0.25 |
| (FIG. 1D) | | | | | | |
| hColon | 2.1.1 | 50 | 1 | 0 | 10 | 0 |
| (FIG. 1D) | | | | | | |
| hColon | 2.1.2 | 50 | 1 | 0 | 10 | 0.05 |
| (FIG. 1D) | | | | | | |
| hColon | 2.1.3 | 50 | 1 | 0 | 10 | 0.1 |
| (FIG. 1D) | | | | | | |
| hColon | 2.1.4 | 50 | 1 | 0 | 10 | 0.25 |
| (FIG. 1D) | | | | | | |
| hColon | 2.2.1 | 50 | 1 | 50 | 0 | 0 |
| (FIG. 1D) | | | | | | |
| hColon | 2.2.2 | 50 | 1 | 50 | 0 | 0.05 |
| (FIG. 1D) | | | | | | |
| hColon | 2.2.3 | 50 | 1 | 50 | 0 | 0.1 |
| (FIG. 1D) | | | | | | |
| hColon | 2.2.4 | 50 | 1 | 50 | 0 | 0.25 |
| (FIG. 1D) | | | | | | |
| hColon | 2.3.1 | 50 | 1 | 5 | 10 | 0 |
| (FIG. 1D) | | | | | | |
| hColon | 2.3.2 | 50 | 1 | 5 | 10 | 0.05 |
| (FIG. 1D) | | | | | | |
| hColon | 2.3.3 | 50 | 1 | 5 | 10 | 0.1 |
| (FIG. 1D) | | | | | | |
| hColon | 2.3.4 | 50 | 1 | 5 | 10 | 0.25 |
| (FIG. 1D) | | | | | | |
| hColon | 2.4.1 | 50 | 1 | 50 | 1 | 0 |
| (FIG. 1D) | | | | | | |
| hColon | 2.4.2 | 50 | 1 | 50 | 1 | 0.05 |
| (FIG. 1D) | | | | | | |
| hColon | 2.4.3 | 50 | 1 | 50 | 1 | 0.1 |
| (FIG. 1D) | | | | | | |
| hColon | 2.4.4 | 50 | 1 | 50 | 1 | 0.25 |
| (FIG. 1D) | | | | | | |
| hSmall | 1.1.1 | 5 | 10 | 0 | 10 | 0 |
| Intestine | | | | | | |
| (FIG. 1G) | | | | | | |
| hSmall | 1.2.1 | 5 | 10 | 50 | 0 | 0 |
| Intestine | | | | | | |
| (FIG. 1G) | | | | | | |
| hSmall | 1.3.1 | 5 | 10 | 5 | 10 | 0 |
| Intestine | | | | | | |
| (FIG. 1G) | | | | | | |
| hSmall | 1.4.1 | 5 | 10 | 50 | 1 | 0 |
| Intestine | | | | | | |
| (FIG. 1G) | | | | | | |
| hSmall | 2.1.1 | 50 | 1 | 0 | 10 | 0 |
| Intestine | | | | | | |
| (FIG. 1G) | | | | | | |
| hSmall | 2.2.1 | 50 | 1 | 50 | 0 | 0 |
| Intestine | | | | | | |
| (FIG. 1G) | | | | | | |
| hSmall | 2.3.1 | 50 | 1 | 5 | 10 | 0 |
| Intestine | | | | | | |
| (FIG. 1G) | | | | | | |
| hSmall | 2.4.1 | 50 | 1 | 50 | 1 | 0 |
| Intestine | | | | | | |
| (FIG. 1G) | | | | | | |

TABLE 2

| Target | Host | Dilution | Duration | Provider |
|---|---|---|---|---|
| Anti-AldolaseB | Rabbit | 1:400 | Overnight at 4° C. | Abcam (ab75751) |
| Anti-Mucin2 | Mouse | 1:400 | Overnight at 4° C. | Santa Cruz (sc-515032) |
| Anti-Yap1 | Rabbit | 1:200 | Overnight at 4° C. | CST (#14074) |
| Anti-NUSAP1 | Rabbit | 1:200 | Overnight at 4° C. | ProteinTech (12024-1-AP) |
| Anti-FABP2 | Goat | 1:200 | Overnight at 4° C. | R&D Systems (AF1486-SP) |
| Anti-Lysozyme | Rabbit | 1:1500 | Overnight at 4° C. | DAKO (A0099) |
| (EC3.2.1.17) | | | | |
| Anti-FABP1 | Mouse | 1:400 | Overnight at 4° C. | R&D Systems (MAB29644-100) |
| Anti-DII1 | Sheep | 1:100 | Overnight at 4° C. | R&D Systems (AF3970) |
| Anti-RANKL | Mouse | 1:200 | Overnight at 4° C. | Abcam (ab45039) |
| Anti-POU2F3 | Rabbit | 1:200 | Overnight at 4° C. | Merck (HPA019652) |
| Anti-REG4 | Rabbit | 1:200 | Overnight at 4° C. | R&D System (AF1379) |
| Anti-OLFM4 | Rabbit | 1:200 | Overnight at 4° C. | CST (#14369S) |
| Anti-Serotonin | Goat | 1:500 | Overnight at 4° C. | Abcam (ab66047) |
| Anti-Chromogranin-A | Rabbit | 1:200 | Overnight at 4° C. | Santa Cruz (sc-13090) |
| Anti-Ki67 | Rabbit | 1:400 | Overnight at 4° C. | Abcam (ab16667) |
| Anti-GFP | Goat | 1:1000 | Overnight at 4° C. | Abcam (ab6556) |
| Anti-Sox9 | Rabbit | 1:200 | Overnight at 4° C. | Abcam (ab185230) |
| Alexa Fluor 488 donkey | Donkey | 1:500 | 3 h at room temp. | Abcam (ab5450) |
| anti rabbit IgG | | | | |
| Alexa Fluor 488 donkey | Donkey | 1:500 | 3 h at room temp. | Thermo Fisher Sci. (A-32814) |
| anti goat IgG | | | | |
| Alexa Fluor 488 donkey | Donkey | 1:500 | 3 h at room temp. | Abcam (ab111125) |
| anti mouse IgG | | | | |
| Alexa Fluor 568 donkey | Donkey | 1:500 | 3 h at room temp. | CST (#2586) |
| anti rabbit IgG | | | | |
| Alexa Fluor 568 donkey | Donkey | 1:500 | 3 h at room temp. | CST (#39141) |
| anti mouse IgG | | | | |
| Alexa Fluor 568 donkey | Donkey | 1:500 | 3 h at room temp. | Abcam (ab175474) |
| anti goat IgG | | | | |

1. A culture medium for the culture of intestinal organoids wherein it comprises neuregulin 1 (NRG1) and all-trans retinoic acid (atRA).

2. The culture medium of claim 1 wherein the intestinal organoids are small intestinal organoids, colon organoids, colorectal cancer organoids, Crohn's disease organoids or Ulcerative Colitis organoids.

3. The culture medium of claim 1 wherein the concentration of NRG1 is between 1 ng/ml and 100 ng/ml and wherein the concentration of atRA is between 10 nM and 10 μM.

4. The culture medium of claim 1 wherein it is free of animal serum.

5. The culture medium of claim 1 further comprising recombinant R-Spondin, recombinant Noggin, NRG1, IGF1, FGF2 and Gastrin.

6. The culture medium of claim 1 further comprising Wnt NGS.

7. A composition comprising a medium according to claim 1 and an extracellular matrix or a 3D matrix that mimics the extracellular matrix by its interactions with cellular membrane proteins.

8. The composition according to claim 7, wherein said 3D matrix extracellular matrix is synthetic hydrogels or is Matrigel™.

9. (canceled)

10. A method of generating intestinal organoids comprising the steps of providing a suspension of single cells or fragments of intestinal tissues incubating said suspension of single cells or fragments of intestinal tissue with a first medium for at least one day, and thereafter exchanging said first medium with a medium according to claim 1.

11. The method of generating intestinal organoids of claim 10 wherein said single cells or fragments of intestinal tissues are obtained from small intestine, colon, colorectal cancer, Crohn's disease tissue, Ulcerative Colitis tissue, or definitive endoderm cells.

12. The method of claim 10 wherein said first medium comprises an inhibitor of the Rho-associated kinase (ROCK) and an inhibitor of the Activin/NODAL/TGF-β pathway.

13. The method of claim 10, wherein the cells are incubated in the first medium for at least three days.

14. An intestinal organoid obtained using the method of claim 10.

15. The method of claim 12 wherein the inhibitor of the Rho-associated kinase (ROCK) is the ROCK inhibitor Y27632.

16. The method of claim 12 wherein the inhibitor of the Activin/NODAL/TGF-β pathway is the inhibitor A 83-01.

17. The method of claim 12 wherein the inhibitor of the Rho-associated kinase (ROCK) is the ROCK inhibitor Y27632 and the inhibitor of the Activin/NODAL/TGF-β pathway is the inhibitor A 83-01.
